(19) United States
(12) Patent Application Publication
(10) Pub. No.: US 2008/0199872 A1
(43) Pub. Date: Aug. 21, 2008

(54) Method and Kit for Analyzing a Target Nucleic Acid Sequence

(75) Inventors: Ross Thomas Barnard, Toowong (AU); Graeme Ross Barnett, Shailer Park (AU)

Correspondence Address: BAKER & DANIELS LLP, 805 15TH STREET, NW, SUITE 700, WASHINGTON, DC, 20005, US

(73) Assignee: Centre for Innovations Pty Ltd, Eight Miles Plains, Queensland (AU)

(21) Appl. No.: 11/994,821

(22) PCT Filed: Jul. 6, 2006
(86) PCT No.: PCT/AU2006/000958

§ 371 Date: Apr. 22, 2008

Related U.S. Application Data
(60) Provisional application No. 60/696,873, filed on Jul. 6, 2005.

(51) Int. Cl. C12Q 1/68

(52) U.S. Cl. 435/6

(57) ABSTRACT

This invention discloses methods for determining the presence of a target nucleic acid sequence using oligonucleotides that cooperate in a nucleic acid processing reaction to produce a detectable signal. In some embodiments, the methods also facilitate quantification of a target nucleic acid sequence. The present invention further discloses kits that can be used to conduct the methods of the present invention.

### FIELD OF THE INVENTION

This invention relates generally to methods for analyzing a target nucleic acid sequence. More particularly, the present invention relates to analytical methods for determining the presence of a target nucleic acid sequence using oligonucleotides that cooperate in a nucleic acid processing reaction to produce a detectable signal. In some embodiments, the methods facilitate quantification of a target nucleic acid sequence. The present invention further relates to kits that can be used in the practice of the methods of the invention.

### BACKGROUND OF THE INVENTION

Nucleic acid molecules may be analysed by using systems that detect hybridization to other short nucleic acid molecules that are commonly referred to as probes, primers or oligonucleotides. This is possible because nucleic acid molecules can be distinguished by their sequences, and by some of their subsequences, and because nucleic acid molecules can bind specifically with sequences, such as oligonucleotides, which are complementary.

The polymerase chain reaction (PCR) is one of the most sensitive of the nucleic acid analysis systems. Hybridization between an oligonucleotide and a complementary target nucleic acid sequence forms a site at which a DNA polymerase initiates polymerization from which it then copies the target nucleic acid template to produce a new DNA strand complementary to the target nucleic acid. DNA is amplified by successive copying of the new strand, as well as the original strand. PCR primer oligonucleotides are incorporated into the amplified product DNA and the amplified product DNA is then identified. Commonly, PCR products are visually detected after separating them by gel electrophoresis and staining them with a chemical. Alternatively, in the absence of gel electrophoresis, PCR products can be detected either quantitatively or semi-quantitatively by preferentially binding a dye to the double stranded PCR products.

Several other nucleic acid analysis methods also exist including ligase chain reaction, oligonucleotide ligation assays, ligation dependent PCR, strand displacement amplification, branched DNA signal amplification, rolling circle amplification, transcription mediated amplification, nucleic acid sequence-based amplification, and hybridization signal amplification.

Despite the existence of numerous nucleic acid analysis methods, however, few methods have been developed that can distinguish between or quantify two or more different nucleic acid targets simultaneously. Generally, methods designed to analyse a number of distinct nucleic acid targets at the same time are unreliable and insensitive. As a result, very few nucleic acid analysis methods which are able to analyse multiple nucleic acid targets simultaneously, i.e., multiplexed, are in routine use in current medical, veterinary or agricultural diagnostics (Yang et al., 2004, Lancet, Infectious Diseases 4: 337-48; Broude et al., 2001, Proc. Natl. Acad. Sci. USA. 98: 206-211; Chamberlain et al., 1988, Nucleic Acids Res. 16: 11141-11156; Edwards et al., 1994, PCR Methods Appl. 3: S65-S75; Hacia et al., 1998, Genome Res. 8: 1245-1258; Li et al., 1996, Nucleic Acids Res. 24: 538-539; Stuven et al., 1996, Pharmacogenetics 6: 417-421; van Orsouw et al., 1998, Genomics 52: 27-36).

Accordingly, it would be highly desirable to develop a sensitive method that is not only capable of analyzing a single target nucleic acid sequence, but is also capable of analyzing multiple target nucleic acid sequences simultaneously, i.e., multiplex analysis.

### SUMMARY OF THE INVENTION

Accordingly, in one aspect, the present invention provides methods for analysing a target nucleic acid sequence in a test sample. These methods generally comprise:
combining in a reaction vessel:
   (1) a capture oligonucleotide (e.g., immobilized or free in solution) that does not hybridize to the target nucleic acid sequence;
   (2) a signaling oligonucleotide that provides a detectable signal and that hybridizes to the capture oligonucleotide;
   (3) at least one chimeric oligonucleotide that comprises:
      (a) a first targeting sequence that hybridizes to a subsequence of the target nucleic acid sequence; and
      (b) a capturable sequence that hybridizes to a sequence selected from:
         (i) the capture oligonucleotide; or
         (ii) the signaling oligonucleotide
   (4) at least one cooperating oligonucleotide that comprises a second targeting sequence that hybridizes to a sequence selected from:
      (a) a different subsequence of the target nucleic acid sequence than the subsequence to which the first targeting sequence hybridizes; or
      (b) a subsequence of a complementary strand of the target nucleic acid sequence, or
      (c) at least one chimeric oligonucleotide; and
   (5) a test sample comprising nucleic acid;
subjecting the contents of the reaction vessel to a nucleic acid processing reaction to form a reaction product if the target nucleic acid sequence is present in the test sample, wherein the reaction product thus formed comprises a first strand comprising at least one chimeric oligonucleotide as well as a second strand comprising at least one cooperating oligonucleotide, or extension product thereof, that blocks the hybridization of the capturable sequence of the chimeric oligonucleotide to the capture oligonucleotide, thereby allowing the signaling oligonucleotide to hybridize to the capture oligonucleotide; and
detecting the detectable signal from the signaling oligonucleotide which indicates the presence or amount of the target nucleic acid sequence in the test sample.

In some embodiments, the nucleic acid processing reaction is a polymerization-dependent nucleic acid processing reaction, illustrative examples of which include:
a) hybridizing the targeting sequence of the chimeric oligonucleotide to the target nucleic acid sequence to form a first hybrid wherein the target nucleic acid sequence extends in a 3′ to 5′ direction beyond the 3′ terminal nucleotide of the chimeric oligonucleotide to define a non-hybrid portion of the target nucleic acid sequence;
b) extending the chimeric oligonucleotide of the first hybrid in the presence of a polymerization agent and nucleotide precursors using the non-hybrid portion of target nucleic acid sequence as a template, to form a first duplex comprising a first extension product and the target nucleic acid sequence;
c) denaturing the first duplex to free the target nucleic acid sequence from the first extension product;
d) hybridizing the cooperating oligonucleotide with the first extension product to form a second hybrid wherein the first extension product extends in a 3′ to 5′ direction beyond the 3′ terminal nucleotide of the cooperating oligonucleotide to define a non-hybrid portion of the first extension product; and
e) extending the cooperating oligonucleotide of the second hybrid in the presence of a polymerization agent and nucleotide precursors using the first extension product as a template, to form a reaction product comprising the first extension product and a second extension product that is complementary to the first extension product.

Suitably, steps a) to e) are repeated one or more times, generally between about 1 and about 100 times, usually between about 10 and about 50 times and more usually between about 20 and about 40 times. In some embodiments, the polymerization agent is a primer dependent DNA polymerase (which is optionally thermostable), illustrative examples of which include Pyrococcus furiosis (Pfu) DNA polymerase, Pyrococcus sp. GB-D (Psp) DNA polymerase, Pyrococcus woesei (Pwo) DNA polymerase, Thermus aquaticus (Taq) DNA polymerase, Thermus brocianus (Tbr) DNA polymerase, Thermus flavus (Tfl) DNA polymerase, Thermococcus litoralis (Tli or Vent) DNA polymerase, Thermotoga maritima (Tma) DNA polymerase and Thermus thermophilus (Tth) DNA polymerase and derivatives thereof.

In some embodiments, the polymerization agent in step b) is a primer dependent reverse transcriptase such as but not limited to avian myeloblastosis virus (AMV), Moloney murine leukemia virus (MMLV) and Thermus thermophilus (Tth) DNA polymerase and derivatives thereof.

In some embodiments, the polymerization agent in step e) is a DNA polymerase, which is suitably thermostable.

In other embodiments, the polymerization-dependent nucleic acid processing reaction comprises:
i) hybridizing a first targeting sequence of a circularizable first cooperating oligonucleotide to a first subsequence of the target nucleic acid sequence to form a first hybrid, wherein the 3′ nucleotide of the first cooperating oligonucleotide is complementary to the 5′ nucleotide of the first subsequence;
ii) hybridizing a second targeting sequence of the first cooperating oligonucleotide to a second subsequence of the target nucleic acid sequence, wherein the second subsequence is located adjacent to the first subsequence, to form a second hybrid,
iii) ligating the first and second targeting sequences of the first cooperating oligonucleotide in the presence of the 5′ nucleotide of the first subsequence and a ligation agent to form a first duplex comprising the first and second subsequences of the target nucleic acid sequence and a ligation product that comprises the first and second targeting sequences of the first cooperating oligonucleotide in a circularized form,
(iv) denaturing the first duplex to free the ligation product from the target nucleic acid,
(v) hybridizing a chimeric oligonucleotide with the ligation product to form a third hybrid;
(vi) extending the chimeric oligonucleotide of the third hybrid in the presence of a polymerization agent and nucleotide precursors using the ligation product as a template, to form a first extension product,
(vii) hybridizing a second cooperating oligonucleotide to the first extension product to form a fourth hybrid, and
(viii) extending the second cooperating oligonucleotide of the fourth hybrid in the presence of a polymerization agent (e.g., a primer dependent DNA polymerase such as but not limited to φ29 DNA polymerase) and nucleotide precursors using the first extension product as a template, to form a reaction product comprising the first extension product and a second extension product that is complementary to the first extension product.

In other embodiments, the nucleic acid processing reaction is a ligase-dependent nucleic acid processing reaction, illustrative examples of which include:
1. hybridizing a first targeting sequence of a chimeric oligonucleotide to a first subsequence of the target nucleic acid sequence to form a first hybrid, wherein the 3′ nucleotide of the chimeric oligonucleotide is complementary to the 5′ nucleotide of the first subsequence;
2. hybridizing the second targeting sequence of a first cooperating oligonucleotide to a second subsequence of the target nucleic acid sequence, wherein the second subsequence is located adjacent to the first subsequence, to form a second hybrid;
3. ligating the chimeric oligonucleotide with the first cooperating oligonucleotide in the presence of the 5′ nucleotide of the first subsequence and a ligation agent to form a first duplex comprising the first and second subsequences of the target nucleic acid sequence and a ligation product that comprises both the chimeric oligonucleotide and the first cooperating oligonucleotide;
4. denaturing the first duplex to free the ligation product from the target nucleic acid; and
5. hybridizing a second cooperating oligonucleotide to the chimeric oligonucleotide and first cooperating sequence portions of the ligation product to form a reaction product.

In other illustrative examples, the ligase-dependent nucleic acid processing reaction comprises:
a. hybridizing a first targeting sequence of a first chimeric oligonucleotide to a first subsequence of the target nucleic acid sequence to form a first hybrid, wherein the 3′ nucleotide of the first chimeric oligonucleotide is complementary to the 5′ nucleotide of the first subsequence and the 5′ nucleotide of the capturable sequence is non-ligatable;
b. hybridizing a second targeting sequence of a second chimeric oligonucleotide to a second subsequence of the target nucleic acid sequence, which second subsequence is adjacent to the first subsequence, to form a second hybrid; wherein the 5′ nucleotide of the capturable sequence is non-ligatable;
c. ligating the first chimeric oligonucleotide with the second chimeric oligonucleotide in the presence of the 5′ nucleotide of the first subsequence and a ligation agent to form a first duplex comprising the first and second subsequences of the target nucleic acid sequence and a ligation product that comprises both the first chimeric oligonucleotide and the second chimeric oligonucleotide;
d. denaturing the first duplex to free the ligation product from the target nucleic acid; and
e. hybridizing a cooperating oligonucleotide to the first and second chimeric oligonucleotides of the ligation product to form a reaction product.

In still other illustrative examples, the ligase-dependent nucleic acid processing reaction comprises:
i. hybridizing a first targeting sequence of a first chimeric oligonucleotide to a first subsequence of the target nucleic acid sequence to form a first hybrid, wherein the first chimeric oligonucleotide comprises a capturable sequence that is capable of hybridizing to the capture oligonucleotide and the 3′ nucleotide of the first chimeric oligonucleotide is complementary to the 5′ nucleotide of the first subsequence;
ii. hybridizing a second targeting sequence of a second chimeric oligonucleotide to a second subsequence of the target nucleic acid sequence, which second subsequence is adjacent to the first subsequence, to form a second hybrid; wherein the first chimeric oligonucleotide comprises a capturable sequence that is capable of hybridizing to the signaling oligonucleotide;
iii. ligating the first chimeric oligonucleotide with the second chimeric oligonucleotide in the presence of the 5′ nucleotide of the first subsequence and a ligation agent to form a first duplex comprising the first and second subsequences of the target nucleic acid sequence and a reaction product that comprises both the first chimeric oligonucleotide and the second chimeric oligonucleotide; and
iv. denaturing the first duplex to free the reaction product from the target nucleic acid.

In some embodiments, steps 1 to 5 or a to e or i to iv are repeated one or more times, generally between about 1 and about 100 times, usually between about 10 and about 50 times and more usually between about 20 and about 40 times.

Suitably, the ligation agent is selected from T4 DNA ligase, Escherichia coli DNA ligase and Thermus filiformis (Tfi) DNA ligase and derivatives thereof.

In another aspect, the present invention provides a kit comprising:
(1) a capture oligonucleotide (e.g., immobilized or free in solution) that does not hybridize to the target nucleic acid sequence;
(2) a signaling oligonucleotide that provides a detectable signal and that hybridizes to the capture oligonucleotide;
(3) at least one chimeric oligonucleotide that comprises:
   (a) a first targeting sequence that hybridizes to a subsequence of the target nucleic acid sequence; and
   (b) a capturable sequence that hybridizes to a sequence selected from:
      (i) the capture oligonucleotide; or
      (ii) the signaling oligonucleotide
(4) at least one cooperating oligonucleotide that comprises a second targeting sequence that hybridizes to a sequence selected from:
   (a) a different subsequence of the target nucleic acid sequence than the subsequence to which the first targeting sequence hybridizes; or
   (b) a subsequence of a complementary strand of the target nucleic acid sequence, or
   (c) at least one chimeric oligonucleotide

In some embodiments, the kit further comprises (5) one or more polymerization and/or ligation agents.

In some embodiments, any one or more of components (1) to (5) (e.g., 1, 2, 3, 4 or 5) are in lyophilized form. Suitably, any two or more of components (1) to (5) (e.g., 2, 3, 4 or 5) are in the form of a mixture. Alternatively, they may be in separate containers.

In some embodiments, the capture oligonucleotide is immobilized on a solid surface (e.g., the surface of a microparticle or bead, a nanowire, a diagnostic strip or reaction vessel).

In some embodiments, two or more capture oligonucleotides are immobilized in the form of a capture oligonucleotide array.

### BRIEF DESCRIPTION OF THE DRAWINGS

FIG. 1: Photographic representation of an agarose gel showing products generated by RT-PCR from PB2 gene segment of Australian Influenza A isolates H5N3(+), H5N3, H11N6, H7N7, H12N9, H7N7, H4N4, H6N5 and H9N2.

FIG. 2: Schematic representation of one embodiment of the method of the present invention using polymerase chain reaction (PCR). (FIG. 2A) Immobilized capture oligonucleotide (B); (FIG. 2B) Signaling oligonucleotide (B″) including a signaling reagent (C); (FIG. 2C) Chimeric oligonucleotide comprising a capturable sequence (B′) and a targeting sequence (E′) & cooperating oligonucleotide (F′); (FIG. 2D) Target nucleic acid sequence (E); (FIG. 2E) Hybrid of chimeric oligonucleotide and target nucleic acid sequence including first extension product (F); (FIG. 2F) Hybrid of chimeric oligonucleotide and target nucleic acid sequence including extension product and cooperating oligonucleotide including second extension product (G); (FIG. 2G) Hybrid of immobilized capture oligonucleotide and signaling oligonucleotide including a signaling reagent (i.e., positive signal); (FIG. 2H) Hybrid of immobilized capture oligonucleotide and chimeric oligonucleotide (i.e., negative signal).

FIG. 3: Schematic representation of one embodiment of the method of the present invention using rolling circle amplification (RCA).

FIG. 4: Schematic representation of one embodiment of the method of the present invention using ligation chain reaction (LCR).

FIG. 5: Schematic representation of one embodiment of the method of the present invention using ligation chain reaction (LCR).

FIG. 6: Schematic representation of one embodiment of the method of the present invention using ligation chain reaction (LCR).

FIG. 7: Graphical representation showing analysis of H7N7 influenza A cDNA using end point detection. Absorbance was measured at 450 nm after 35 cycles of PCR, using 0.5 pmole of PCR-TAG primer in 50 μL.

FIG. 8: Graphical representation showing the results of a titration experiment to determine the optimal amount of PCR-TAG primer in one embodiment of the method of the present invention, which uses an end point detection step. Absorbance of the samples was measured at 450 nm after 35 cycles of PCR amplification. The darker points show absorbance when PCR target is present and the lighter points show background absorbance.

FIG. 9: Photographic representation showing agarose gel electrophoresis and ethidium bromide staining of PCR products amplified according to the same assay referenced in FIG. 8, to determine the optimal amount of PCR-TAG primer. In each case the reverse primer was used at 4 times the amount of the PCR-TAG primer. Negative controls contained no starting template and the total PCR reaction volume was 50 μL. Lane numbers designate the following: M, Hyper Ladder II; (1) PCR product using 1 pmole of PCR-TAG primer and H7N7 template; (2) PCR product using 0.5 p mole of PCR-TAG primer and H7N7 template; (3) PCR product using 0.25 p mole of PCR-TAG primer and H7N7 template; (4) PCR product using 1 p mole of PCR-TAG primer negative control; (5) PCR product using 0.5 p mole of PCR-TAG primer negative control; and (6) PCR product using 0.25 p mole of PCR-TAG primer negative control.

### BRIEF DESCRIPTION OF THE SEQUENCES

| SEQ | |
|---|---|
| ID | |
| NO | SEQUENCE |
| 1 | 5′-CTT TAA TCT CAA TCA ATA CAA ATC-3′ |
| 2 | 5′-CTT TAA TCT CAA TCA ATA CAA ATC AG(C/T) TCI |
| | TC(C/T) TT(C/T) AG(C/T) TT(C/T) GG-3′ |
| 3 | 5′-AGT AT(T/C) CTC AT(T/C) CC(T/A) GAN CC-3′ |
| 4 | 5′-GAT TTG TAT TGA TTG AGA TTA AAG-3′ |
| 5 | 5′-CTT TAA TCT CAA TCA ATA CAA ATC TTG |
| | CAG CIG CIC CAC CIG-3′ |
| 6 | 5′-AIC AIA GIA GIA TGC AGT-3′ |
| 7 | 5′-AIC AIA GIA GIA TGC AGT TCA TAA GAC TCG TCA |
| | TGT CTC AGC AGC TTC TAA CGG TCA CTA ATA CGA CTC |
| | ACT ATA GG TTG CAG CIG CIC CAC CIG-3′ |
| 8 | 5′-CTT TAA TCT CAA TCA ATA CAA ATC GCT GAG ACA |
| | TGA CGA GTC-3′ |
| 9 | 5′-AAT ACG ACT CAC TAT AGG-3′ |
| 10 | 5′-CTT TAA TCT CAA TCA ATA CAA ATC GAI GTI AGI |
| | GAI AGI CAI GG-3′ |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

The terms “amplification” or “nucleic acid amplification” or “amplification reaction” refers to a biochemical reaction that produces many polynucleotide copies of a particular target nucleic acid sequence. If the target nucleic acid sequence is single-stranded complementary sequences may be produced in the reaction. In some embodiments, the reaction is a polymerase chain reaction (PCR) or a similar reaction that uses a polymerase to copy a nucleic acid sequence such as helicase-dependent amplification (HDA), transcription mediated amplification (TMA), strand displacement amplification (SDA), nucleic acid sequence-based amplification (NASBA), rolling circle amplification (RCA) and reverse transcription polymerase chain reaction (RT-PCR). A double stranded region formed through the hybridization of an oligonucleotide to a single-stranded form of the target nucleic acid sequence is required to prime (start) the reaction. In other embodiments, the terms “amplification” or “nucleic acid amplification” or “amplification reaction” refer to a biochemical reaction using a ligase or similar enzyme that covalently links two oligonucleotides or two oligonucleotide sub-sequences, such as a ligase chain reaction (LCR). Ligase enzymes ligate the two oligonucleotides or oligonucleotide sub-sequences when they hybridize at adjacent sites in the target nucleic acid sequence. Alternatively, if the two oligonucleotides or oligonucleotide subsequences hybridize at sites that are one or more nucleic acid residues apart, i.e., they are not adjacent, then the single stranded region between the double stranded regions is converted to a double stranded region using a polymerase, and the ligase enzyme then links the adjacent oligonucleotides to form a continuous double stranded region.

The term “capturable sequence” refers to a nucleic acid sequence that is capable of hybridizing with another nucleic acid sequence. In some embodiments, the capturable sequence hybridizes to a capture oligonucleotide which in illustrative examples is immobilized to a support (e.g., a solid surface) or free in solution.

The term “capture oligonucleotide array” means a plurality of capture oligonucleotides immobilized at discrete known locations on a solid surface. In relation to the surface of a reaction vessel or diagnostic strip, the capture oligonucleotides may be arranged in a two-dimensional spatially addressed array, e.g., a 2×2 array. Alternatively, the capture oligonucleotides may be arranged in a tubular array in which a two-dimensional planar sheet is rolled into a three-dimensional tubular configuration. In other embodiments, the capture oligonucleotides are arranged on the inner or outer surface of a two- or three-dimensional reaction vessel of any convenient topology. Nucleic acids arrays are known in the art, and can be classified in a number of ways; both ordered arrays (e.g., the ability to resolve chemistries at discrete sites), and random arrays are included. Ordered arrays include, but are not limited to, those made using photolithography techniques (Affymetrix GeneChip™), spotting techniques (Synteni and others), printing techniques (Hewlett Packard and Rosetta), three dimensional “gel pad” arrays, etc. Liquid arrays may also be used, i.e., three-dimensional array methods such as flow cytometry. When flow cytometry is used, capture oligonucleotides are suitably immobilized to a support such as a microsphere.

In some embodiments, the ordered arrays include arrays that contain nucleic acids at known locations. That is, the capturable sequences or capture oligonucleotides described herein are immobilized at known locations on a substrate. By “known” locations is meant a site that is known or has been known.

The term “chimeric oligonucleotide” is used herein to refer to an oligonucleotide comprising at least two nucleic acid sequences or portions that are positioned or linked in a manner that does not normally occur in nature.

The terms “complementary” and “complementarity” refer to a sequence of nucleotides related by the base-pairing rules. For example, the sequence “A-G-T-C” is complementary to the sequence “T-C-A-G”. Complementarity may be “partial,” in which only some of the nucleic acids' bases are matched according to the base pairing rules. As such, the complementarity need not be perfect; there may be any number of base pair mismatches that will interfere with hybridization between a first sequence and a second sequence. However, if the number of mutations is so great that no hybridization can occur under even the least stringent of hybridization conditions, the sequence is not a complementary sequence. Thus, by “substantially complementary” is meant that a first sequence is sufficiently complementary to a second sequence to hybridize under the selected reaction conditions. The relationship of complementarity and stringency of hybridization sufficient to achieve specificity is well known in the art. Therefore, substantially complementary sequences can be used in any of the analysis methods of the present invention. Such sequences can be, for example, perfectly complementary or can contain from 1 to many mismatches so long as the hybridization conditions are sufficient to allow discrimination between a target sequence and a non-target sequence. Accordingly, substantially complementary sequences can range in percent identity from 100, 99, 98, 97, 96, 95, 94, 93, 92, 91, 90, 89, 85, 80, 75 or less. Alternatively, there may be “complete” or “total” complementarity between the nucleic acids. The degree of complementarity between nucleic acid strands has significant effects on the efficiency and strength of hybridization between nucleic acid strands.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

“Hybridization” is used herein to denote the pairing of complementary nucleotide sequences to produce a DNA-DNA, DNA-RNA or a DNA-PNA hybrid. Complementary base sequences are those sequences that are related by the base-pairing rules. In relation to DNA, A pairs with T and C pairs with G. In relation to RNA U pairs with A and C pairs with G. The base inosine (I) may also be used. Inosine can form base pairs with C or A or G or T (in descending order of stability). In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

By “isolated” is meant material that is substantially or essentially free from components that normally accompany it in its native state. For example, an “isolated oligonucleotide,” as used herein, refers to an oligonucleotide, which has been purified from the sequences that flank it in a naturally occurring state, e.g., a DNA fragment that has been removed from the sequences that are normally adjacent to the fragment.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

The terms “oligonucleotide”, “polynucleotide” or “nucleic acid” as used herein designate DNA, cDNA, RNA, mRNA, cRNA or PNA. The term typically refers to oligonucleotides greater than 30 nucleotides in length.

By “primer” is meant an oligonucleotide which, when paired with a strand of DNA or RNA, is capable of initiating the synthesis of a primer extension product in the presence of a suitable polymerizing agent. The primer is typically single-stranded for maximum efficiency in amplification but may alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridize and serve as a site for the initiation of synthesis. By “substantially complementary”, it is meant that the primer is sufficiently complementary to hybridize with a target nucleotide sequence. Suitably, the primer contains no mismatches with the template to which it is designed to hybridize but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridize therewith and thereby form a template for synthesis of the extension product of the primer.

Terms used to describe sequence relationships between two or more nucleic acid sequences include “reference sequence,” “comparison window,” “sequence identity,” “percentage of sequence identity” and “substantial identity”. A “reference sequence” is at least 10 but frequently 15 to 20 and often at least 25 monomer units, i.e., nucleotides, in length. Because two nucleic acid sequences may each comprise (1) a sequence (i.e., only a portion of the complete nucleotide sequence) that is similar between the two polynucleotides, and (2) a sequence that is divergent between the two nucleic acid sequences, sequence comparisons between two (or more) nucleic acid sequences are typically performed by comparing sequences of the nucleic acid sequences over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “reaction vessel” refers to a container in which the method of the present invention is carried out. The reaction vessel may be any vessel suitable for use with standard molecular biology reactions, and therefore, will be constructed from material that is suitable for such use. Such material may be natural or synthetic, or may be formed from a combination of natural and synthetic materials. Illustrative materials from which a reaction vessel can be constructed include plastic (e.g., polycarbonate, polystyrene and polypropylene), glass and the like. Particularly preferred reaction vessels of the present invention include conventional PCR tubes and microplates e.g., 96-well microplates. In some embodiments of the present invention, the capture oligonucleotides are immobilized on an internal surface of the reaction vessel. In these embodiments, the reaction vessel is suitably constructed of a transparent material such that hybridization of the signaling oligonucleotide to the capture oligonucleotide can be detected from outside the reaction vessel. In specific embodiments, the reaction vessel is characterized in that it has a surface that is substantially planar. Alternatively, the reaction vessel may be characterized in that it has a surface that is substantially tubular in which a two dimensional planar sheet is rolled into a three dimensional tubular configuration. Such configuration could be used to immobilize a greater surface area of capture oligonucleotides into, for example, a “flow through” cell. In other embodiments, the reaction vessel is a microfluidic device

The terms “sequence identity” and “identity” are used interchangeably herein to refer to the extent that nucleic acid sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridization. The higher the stringency, the higher will be the degree of complementarity between hybridized nucleic acid sequences.

“Stringent conditions” as used herein refers to temperature and ionic conditions under which only polynucleotides and oligonucleotides that are substantially complementary or having a high proportion of complementary bases, preferably having exact complementarity, will hybridize and, in some embodiments, yield amplification products. The stringency required is nucleotide sequence dependent and depends upon the various components present during hybridization, and is greatly changed when nucleotide analogues are used. Stringent conditions are well known to those of skill in the art. Generally, for oligonucleotides used as probes in hybridization reactions stringent conditions are selected to be about 10 to 20° C. less than the calculated thermal melting point (Tm) for the specific sequence at a defined ionic strength and pH. The Tm is the temperature (under defined ionic strength and pH) at which 50% of a target sequence hybridizes to a complementary probe. Tm calculations are well known to those expert in the art. Tm is best calculated for oligonucleotides less than 14 base residues long by using the formulae Tm=4(wG+xC)+2(yA+zT)−16.6*log 10(0.050)+16.6*log 10([Na+]), where w, x, y, z are the number of the bases G, C, A, T in the sequence, respectively, and [Na+] is the salt concentration. For oligonucleotides longer than 13 base residues, the Tm is calculated by the nearest neighbour formulae as described by Breslauer et al., (1986, Proc. Nat. Acad. Sci. 83:3746-50), but using the values published by Sugimoto et al., (1996, Nucl. Acids Res, 24:4501-4505). Values for RNA thermodynamic properties can be taken from Xia et al. (1998, Biochemistry 37:14719-14735). It will be understood that an oligonucleotide probe or primer will hybridize to a target sequence under at least low stringency conditions, preferably under at least medium stringency conditions and more preferably under high stringency conditions. Reference herein to low stringency conditions for probe hybridization reactions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. Medium stringency conditions for probe hybridization reactions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaBPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 42° C. High stringency conditions for probe hybridization reactions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. Other stringent conditions for probe hybridization reactions are well known in the art. A skilled addressee will recognise that various factors can be manipulated to optimise the specificity of the hybridization. Optimisation of the stringency of the final washes can serve to ensure a high degree of hybridization. For detailed examples, see CURRENT PROTOCOLS IN MOLECULAR BIOLOGY (supra) at pages 2.10.1 to 2.10.16 and MOLECULAR CLONING. A LABORATORY MANUAL (Sambrook, et al., eds.) (Cold Spring Harbor Press 1989) at sections 1.101 to 1.104.

The phrase “target nucleic acid sequence” refers to any nucleic acid sequence of interest. It may be an entire gene, or portion thereof. As such, the target nucleic acid sequence may be a portion of a gene comprising a genetic mutation such as, but not limited to, nucleotide insertions, deletions and single nucleotide polymorphisms (SNPs). The target nucleic acid sequence may also be a nucleic acid encoded by an entire gene, or portion thereof. The target nucleic acid sequences contemplated by the present invention include, therefore, DNA, cDNA, RNA, mRNA and cRNA. The target nucleic acid sequence may also be a naturally occurring or synthetic nucleic acid molecule.

### 2. Method for Analysing a Target Nucleic Sequence

The present invention provides methods for analysing, e.g., determining the presence or amount of, a target nucleic acid sequence. The method uses a plurality of oligonucleotides that cooperate in a nucleic acid processing reaction to produce a detectable signal in the presence of a target nucleic acid sequence. The plurality of oligonucleotides comprises:
(1) a capture oligonucleotide that is optionally immobilized on a solid surface and that does not hybridize to the target nucleic acid sequence;
(2) a signaling oligonucleotide that provides a detectable signal and that hybridizes to the capture oligonucleotide;
(3) at least one chimeric oligonucleotide, which in some embodiments can function as a primer, that comprises:
   (a) a first targeting sequence that hybridizes to a subsequence of the target nucleic acid sequence; and
   (b) a capturable sequence that hybridizes to a sequence selected from:
      (i) the capture oligonucleotide; or
      (ii) the signaling oligonucleotide; and
(4) at least one cooperating oligonucleotide, which in some embodiments can function as a primer, that comprises a second targeting sequence that hybridizes to a sequence selected from:
   (a) a different subsequence of the target nucleic acid sequence than the subsequence to which the first targeting sequence hybridizes; or
   (b) a subsequence of a complementary strand of the target nucleic acid sequence, or
   (c) at least one chimeric oligonucleotide.

The nucleic acid processing reaction of the present invention results in a reaction product that comprises a first strand comprising at least one chimeric oligonucleotide as well as a second strand comprising at least one cooperating oligonucleotide, or an extension product thereof, that blocks the hybridization of the capturable sequence of the chimeric oligonucleotide to the capture oligonucleotide, thereby allowing the signaling oligonucleotide to hybridize to the capture oligonucleotide and signal the presence or amount of the target. When the target nucleic acid sequence is not present, the nucleic acid processing reaction cannot occur, thereby allowing the capturable sequence of the chimeric oligonucleotide to hybridize to the capture oligonucleotide and block signaling.

### 3. Nucleic Acid Processing Reaction

Any nucleic acid processing reaction that results in the generation of a reaction product as described above may be used in accordance with the method of the present invention. Illustrative nucleic acid processing reactions include nucleic acid amplification. Representative methods for nucleic acid amplification are well known in the art, and include, but are not limited to, PCR (see, e.g., Saiki et al, 1985, Science, 230: 1350-1354; Mullis et al., 1987, Methods Enzymol 155: 335-350), Strand Displacement Amplification (SDA and multiple SDA (MSDA); see, e.g., U.S. Pat. No. 5,422,252 and Little et al), Rolling Circle Amplification (RCA; see, e.g., Liu et al., 1996, J. Am. Chem. Soc 118: 1587-1594 and U.S. Pat. No. 5,854,033 and U.S. Pat. No. 6,642,034), Nucleic Acid Sequence Based Amplification (NASBA; see, e.g., Sooknanan et al., 1994, Biotechniques 17: 1077-1080), Ligase Chain Reaction (LCR; see, e.g., WO 89/09835) and Qβ Replicase Amplification (see, e.g., Tyagi et al, 1996, supra). Various permutations on these techniques may also be employed as reviewed by Syvänen Anne-Christine, (2001, supra). In the present method, any useful combination of features of different amplification reactions may be used to increase the sensitivity and/or specificity of the method.

In some embodiments, the nucleic acid processing reaction of the present invention is based on polymerase dependent nucleic acid amplification. An illustrative amplification of this type is polymerase chain reaction (PCR), in which an extension product synthesised from one oligonucleotide of an individual oligonucleotide pair, when separated from its complement, can serve as a template for synthesis of an extension product of the other oligonucleotide of the pair. Generally, a thermostable primer dependent polymerase is employed, however, as would be known by one skilled in the art, the choice of polymerase is generally dependent upon the particular PCR method used.

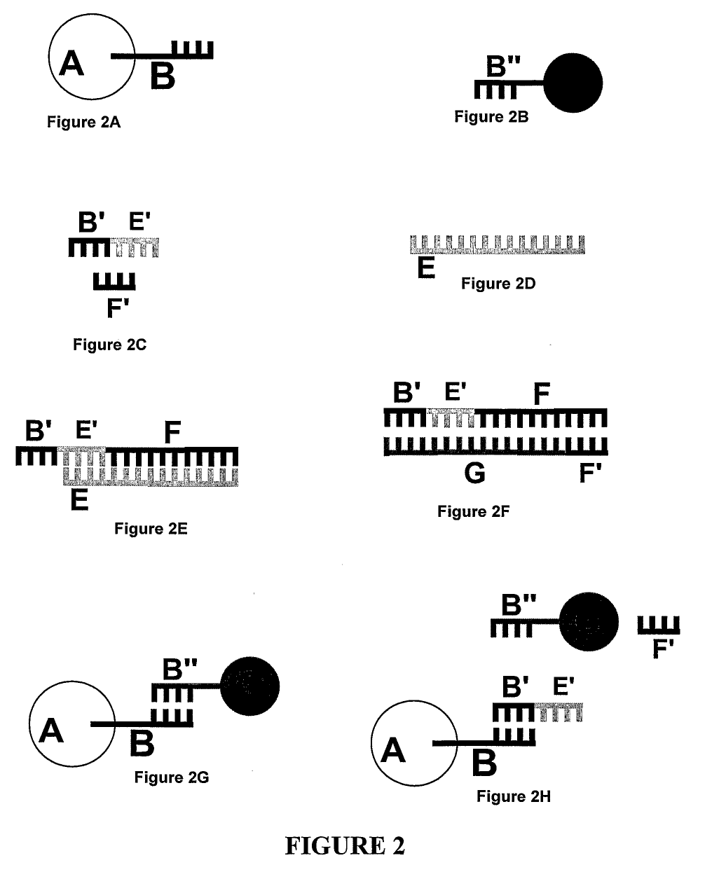

In illustrative examples of this type, such as that shown in FIG. 2, a first hybrid is formed between the targeting sequence of the chimeric oligonucleotide and the target nucleic acid sequence. The targeting sequence is then extended with a polymerization agent, which may be a primer-dependent DNA polymerase or a primer dependent reverse transcriptase. Such enzymes have the effect of incorporating nucleoside triphosphates (e.g., deoxyribonucleotide triphosphates; dNTPs) into an extension of the targeting sequence of the hybrid, either selectively for hybrids that contain perfectly matched pairings between the 3′ terminal nucleotide of the oligonucleotide primer and the 5′ terminal nucleotide of the target nucleotide sequence or non-selectively whether or not such pairing is perfectly matched. In this regard, it is well known that some enzymes such as, for example, eukaryotic primer-dependent DNA polymerases and avian myeloma virus (AMV) reverse transcriptase have no 3′ error-correcting activity (exonuclease⁻ (exo⁻)), and therefore will extend only oligonucleotides which are bound in hybrids containing a perfect match between the 3′ terminal nucleotide of the oligonucleotide and the 5′ terminal nucleotide of the target nucleotide sequence. Alternatively, other polymerization agents, such as primer dependent DNA polymerases of prokaryotic origin including, for example, the Klenow fragment of Escherichia coli DNA polymerase I, have an error-correcting activity (exonuclease⁺ (exo⁺)), and therefore do not show this selectivity although modifications of such enzymes are available from commercial suppliers wherein such error-correcting activity is absent. However, it is generally desirable to employ 3′ exo⁻ polymerization agents for oligonucleotide extension. Suitable polymerization agents which may be utilized in accordance with PCR based nucleic acid processing reactions would be well known to a person skilled in the art. In order to allow repeated cycling of PCR without the need to add further DNA polymerase, the DNA polymerases are suitably thermostable and include, but are not limited to Pyrococcus furiosis (Pfu) DNA polymerase, Pyrococcus sp. GB-D (Psp) DNA polymerase, Pyrococcus woesei (Pwo) DNA polymerase, Thermus aquaticus (Taq) DNA polymerase, Thermus brocianus (Tbr) DNA polymerase, Thermus flavus (Tfl) DNA polymerase, Thermococcus litoralis (Tli or Vent) DNA polymerase, Thermotoga maritima (Tma) DNA polymerase and Thermus thermophilus (Tth) DNA polymerase and derivatives thereof. Suitable reverse transcriptases that may be used in accordance with the present invention include, but are not limited to, avian myeloblastosis virus (AMV), Moloney murine leukemia virus (MMLV) and Thermus thermophilus (Tth) DNA polymerase and derivatives thereof. Other factors in selecting the polymerization agent include whether the nucleic acid in the test sample is DNA or RNA (i.e., typically only reverse transcriptases will effectively incorporate deoxynucleoside triphosphates into an extension product on an RNA template).

The extension of the targeting sequence of the chimeric oligonucleotide, using the target nucleic acid sequence as a template, results in a duplex comprising the first extension product and the target nucleic acid sequence. The first extension product is then separated from the target nucleic acid sequence by denaturation such that a cooperating oligonucleotide can hybridize to the first extension product, thus forming a second hybrid. Next, the cooperating oligonucleotide is extended, as described above, using the first extension product as a template. As the first extension product comprises the chimeric oligonucleotide, the second duplex formed at this stage comprises the first extension product and a second extension product that is complementary to the first extension product and therefore, to the capturable sequence of the chimeric oligonucleotide. This second duplex corresponds to a reaction product in accordance with the present invention. The sequence that is complementary to the capturable sequence is a “blocking” sequence, which serves to block the hybridization of the capturable sequence of the chimeric oligonucleotide to the capture oligonucleotide, thereby allowing the signaling oligonucleotide to hybridize to the capture oligonucleotide.

In other embodiments, the polymerase dependent amplification comprises rolling circle amplification (RCA), in which hybridization of oligonucleotide primers to a circular nucleic acid molecule permits ligation, i.e., circularization, and a DNA polymerase, typically one that has strand displacement activity, to synthesize a first extension product using the circular nucleic acid molecule as a template. Generally, the extension product is a long nucleic acid molecule containing multiple repeats of sequences complementary to the template circular nucleic acid molecule, In the presence of a complementary oligonucleotide primer, the first extension product can then serve as a template for the synthesis of further extension products, apropos of PCR, thereby permitting amplification of the original template circular nucleic acid molecule.

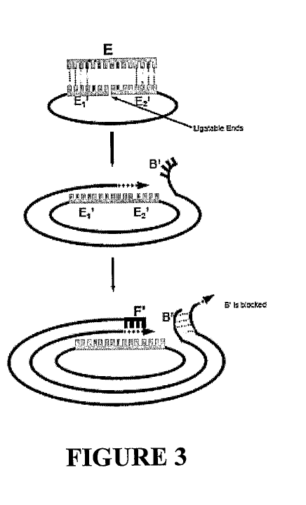

In illustrative examples of this type, such as shown in FIG. 3, a first hybrid is formed between a cooperating oligonucleotide and the target nucleic acid sequence so that the cooperating oligonucleotide is circularized upon hybridization. The cooperating oligonucleotide has ligatable ends (E₁′ and E₂′ as shown in FIG. 3) such that ligation of the 5′ and 3′ ends of the cooperating oligonucleotide can be catalyzed by a ligase enzyme (described in more detail below). A chimeric oligonucleotide, which comprises a capturable sequence (B′), hybridizes to the circularized cooperating oligonucleotide and primes the production of a first extension product catalyzed by a strand displacing polymerase. Another cooperating oligonucleotide (F′), which is complementary to a region of the first extension product, is added and primes the generation of a second extension product provided that ligation of the circular probe has occurred. The second extension product is in part complementary to the capturable sequence of the chimeric oligonucleotide. The sequence in the second extension product that is complementary to the capturable sequence is a “blocking” sequence, which blocks the hybridization of the capturable sequence of the chimeric oligonucleotide to the capture oligonucleotide, thereby allowing the signaling oligonucleotide to hybridize to the capture oligonucleotide.

DNA polymerases suitable for RCA as contemplated by the present invention are suitably capable of displacing the strand complementary to the template strand, termed strand displacement, and lack a 5′ to 3′ exonuclease activity. Strand displacement is necessary to result in synthesis of multiple copies of the ligated cooperating oligonucleotide. A 5′ to 3′ exonuclease activity, if present, might result in the destruction of the synthesized strand. It is also desirable that DNA polymerases for use in the disclosed method are highly processive. The suitability of a DNA polymerase for use in the disclosed method can be readily determined by assessing its ability to carry out rolling circle replication. Exemplary rolling circle DNA polymerases include bacteriophage Φ29 DNA polymerase (U.S. Pat. Nos. 5,198,543 and 5,001,050.), phage M2 DNA polymerase (Matsumoto et al., Gene 84:247 (1989)), phage ΦPRD1 DNA polymerase (Jung et al., Proc. Natl. Acad. Sci. USA 84:8287 (1987)), VENT® DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), and T4 DNA polymerase holoenzyme (Kaboord and Benkovic, Curr. Biol. 5:149-157 (1995)). In specific embodiments, Φ29 DNA polymerase is employed.

Strand displacement can be facilitated through the use of a strand displacement factor, such as helicase. It is considered that any DNA polymerase that can perform rolling circle replication in the presence of a strand displacement factor is suitable for use in the disclosed method, even if the DNA polymerase does not perform rolling circle replication in the absence of such a factor. Strand displacement factors useful in RCA include, but are not restricted to, BMRF1 polymerase accessory subunit (Tsurumi et al., J. Virology 67:7648-7653 (1993)), adenovirus DNA-binding protein (Zijderveld and van der Vliet, J. Virology 68:1158-1164 (1994)), herpes simplex viral protein ICP8 (Boehmer and Lehman, J. Virology 67:711-715 (1993); Skaliter and Lehman, Proc. Natl. Acad. Sci. USA 91:10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

In other embodiments, the nucleic acid processing reaction is based on ligase dependent nucleic acid amplification. Oligonucleotide ligation assays are described, in particular, in U.S. Pat. No. 4,883,750. One example of ligase-dependent reaction is ligase chain reaction (LCR), in which one oligonucleotide hybridizes to a first target sequence and another oligonucleotide hybridizes to a second target sequence that is adjacent to the first target sequence. The hybridized pair of oligonucleotides serves as substrates for ligation to produce a ligation product that comprises both oligonucleotides. If required, the ligation product can then be displaced from the target sequence, e.g., by denaturation, to permit the production of further ligation products from the target sequence, thus resulting in amplification of the ligated oligonucleotides that are complementary to the target nucleic acid.

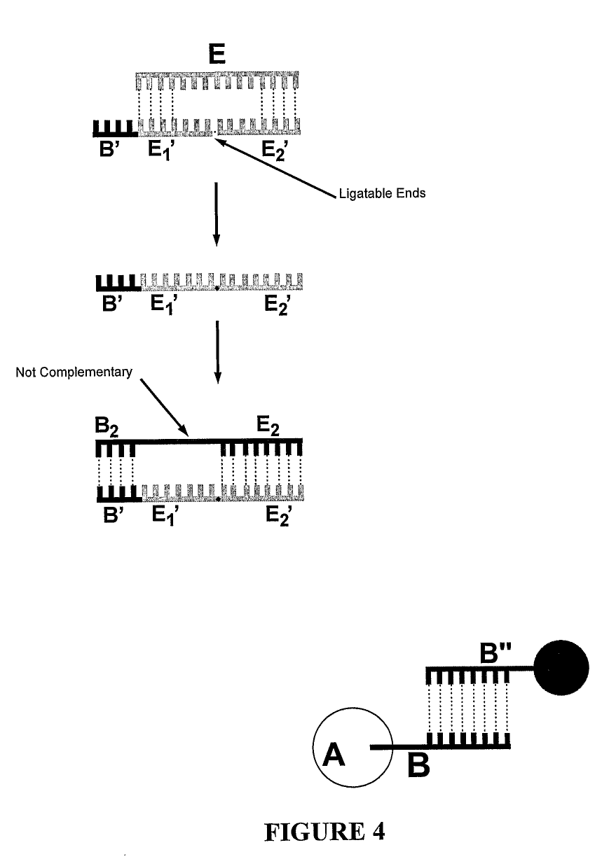

In illustrative examples of this type, such as shown in FIG. 4, a first hybrid is formed by hybridizing a first targeting sequence of a chimeric oligonucleotide to a first subsequence of the target nucleic acid sequence, wherein the 3′ nucleotide of the chimeric oligonucleotide is complementary to the 5′ nucleotide of the first subsequence. A second hybrid is then formed by hybridizing a second targeting sequence of a first cooperating oligonucleotide to a second subsequence of the target nucleic acid sequence, wherein the second subsequence is located adjacent to the first subsequence. The chimeric oligonucleotide is then ligated with the first cooperating oligonucleotide in the presence of the 5′ nucleotide of the first subsequence and a ligation agent to form a first duplex comprising the first and second subsequences of the target nucleic acid sequence and a ligation product that comprises both the chimeric oligonucleotide and the first cooperating oligonucleotide. Suitable ligation agents, which may be utilized in these embodiments, are well known to persons skilled in the art and include, but are not limited to, T4 DNA ligase, Escherichia coli DNA ligase and Thermus filiformis (Tfi) DNA ligase and derivatives thereof. In certain embodiments, the ligation agent is thermostable. Following ligation, the ligation product is separated from the target nucleic acid sequence by denaturation, such that a second cooperating oligonucleotide can hybridize to the chimeric oligonucleotide and first cooperating sequence portions of the ligation product to form a reaction product. The second cooperating oligonucleotide has a blocking region that is complementary to the capturable sequence of the chimeric oligonucleotide, i.e., a blocking sequence, which serves to block the hybridization of the capturable sequence of the chimeric oligonucleotide to the capture oligonucleotide, thereby allowing the signaling oligonucleotide to hybridize to the capture oligonucleotide. Typically, when the first cooperating oligonucleotide has not ligated to the chimeric oligonucleotide, i.e., when the target nucleic acid sequence is not present, the blocking region of the second cooperating oligonucleotide will not hybridize, or will only hybridize with relatively low affinity, to the capturable sequence. As would be known to persons skilled in the art, this is possible by (i) adjusting the size of the blocking region of the second cooperating oligonucleotide and/or (ii) the complementarity of that blocking region to the capturable sequence of the chimeric oligonucleotide; or (iii) by insertion of non-complementary sequences adjacent to the blocking sequence such that substantial hybridization of the blocking region to the capturable sequence only occurs when the first cooperating oligonucleotide has ligated to the chimeric oligonucleotide.

Adjustment of the base composition of the non-complementary sequence adjacent to the blocking sequence so that is more or less complementary to the first ligation product can modulate the sensitivity of the detection system in the following manner. If the non-complementary sequence (the segment between B₂ and E₂ in FIG. 4) is adjusted (or “tuned”) to be more complementary to the first ligation product it will increase the sensitivity of the detection system (i.e., render it able to detect lower concentrations of the target sequence), whereas if it is adjusted (or “tuned”) to be even less complementary to the first ligation product, then the detection system will be less sensitive. In this way, the tunable non-complementary intervening sequence can be used to modulate the sensitivity of the processing reaction.

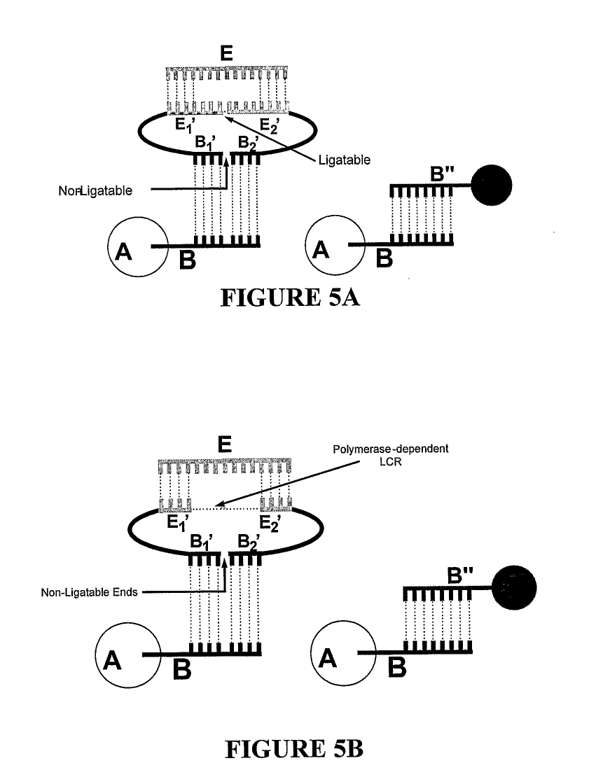

Another illustrative example of the use of LCR is shown in FIG. 5A and comprises hybridizing a first targeting sequence of a first chimeric oligonucleotide (E₁′-B₁′) to a first subsequence of the target nucleic acid sequence to form a first hybrid, wherein the 3′ nucleotide of the first chimeric oligonucleotide is complementary to the 5′ nucleotide of the first subsequence and the 5′ nucleotide of the capturable sequence (the capturable sequence being part of the first chimeric oligonucleotide) is non-ligatable. This is followed by the hybridization of a second targeting sequence (E₂′-B₂′) of a second chimeric oligonucleotide to a second subsequence of the target nucleic acid sequence, which second subsequence is adjacent to the first subsequence, to form a second hybrid; wherein the 3′ nucleotide of the capturable sequence (the capturable sequence being part of the second chimeric oligonucleotide) is non-ligatable. The first chimeric oligonucleotide is ligated with the second chimeric oligonucleotide in the presence of the 5′ nucleotide of the first subsequence and a ligation agent to form a first duplex comprising the first and second subsequences of the target nucleic acid sequence and a ligation product that comprises both the first chimeric oligonucleotide and the second chimeric oligonucleotide. Suitable ligation agents, which may be utilized in this embodiment, are well known to persons skilled in the art and include, but are not limited to, T4 DNA ligase, Escherichia coli DNA ligase and Thermus filiformis (Tfi) DNA ligase and derivatives thereof. In some embodiments, the ligation agent is thermostable. The first duplex is then denatured to free the ligation product from the target nucleic acid. In this manner the ligation product is available to hybridize to a capture oligonucleotide (B in FIG. 5A), thereby competing for the binding of a signaling oligonucleotide (B″ in FIG. 5A). In this example, the amount of signal will over a predetermined range be inversely related to the quantity of starting target sequence. In a modification of this example, there is a “gap” between the target sequences to which the first chimeric probe (E1′-B1′) and the second chimeric probe (E2′-B2′) hybridize (FIG. 5B). After hybridization a polymerase is used to fill in the gap between E1′ and E2′ prior to the ligation step catalyzed by a ligase enzyme. Alternatively a gap filling oligonucleotide that is complementary to the gap sequence in the target (E) is added during the hybridization step and it participates in the ligation reaction.

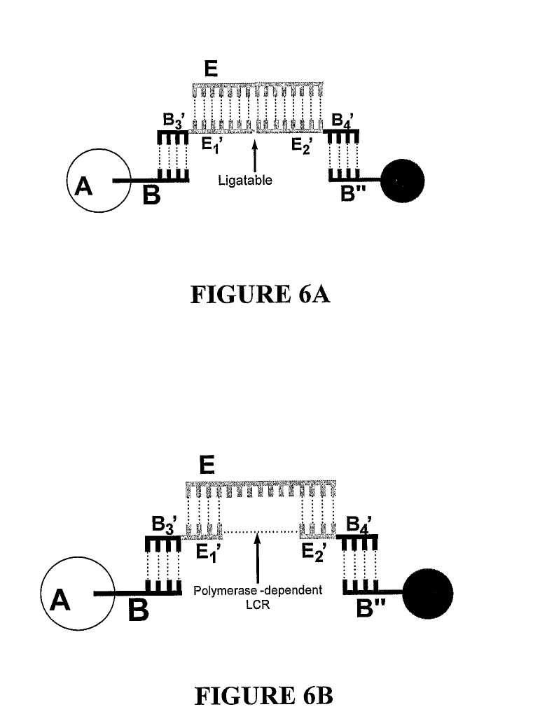

Another illustrative example of the use of LCR is shown in FIG. 6A and comprises hybridizing a first targeting sequence of a first chimeric oligonucleotide to a first subsequence of the target nucleic acid sequence (E) to form a first hybrid, wherein the first chimeric oligonucleotide comprises a capturable sequence (B₃′) that is capable of hybridizing to the capture oligonucleotide and the 3′ nucleotide of the first chimeric oligonucleotide is complementary to the 5′ nucleotide of the first subsequence. A second targeting sequence of a second chimeric oligonucleotide (E₂′-B₄′) is hybridized to a second subsequence of the target nucleic acid sequence (E), which second subsequence is adjacent to the first subsequence, to form a second hybrid; wherein the second chimeric oligonucleotide comprises a capturable sequence (B4′) that is capable of hybridizing to the signaling oligonucleotide (B″). The first chimeric oligonucleotide is ligated with the second chimeric oligonucleotide in the presence of the 5′ nucleotide of the first subsequence and a ligation agent to form a first duplex comprising the first and second subsequences of the target nucleic acid sequence and a reaction product that comprises both the first chimeric oligonucleotide and the second chimeric oligonucleotide. The first duplex is denatured to free the reaction product from the target nucleic acid. The reaction product is detected by hybridization with a capture oligonucleotide on a substrate and a signaling oligonucleotide that contains a detectable moiety such as, but not limited to, a latex bead, colloidal gold, an enzyme, quantum dot or signal amplification technology (SAT; e.g., U.S. Pat. No. 5,902,724). In this latter example, the amount of signal will, over a predetermined range, be proportional to the quantity of target sequence in the initial sample. A related embodiment (FIG. 6B) requires the presence of a polymerase to fill the gap sequence between the regions complementary to E1′ and E2′ or the use of a ligatable gap filling oligonucleotide with suitable phosphorylated ends to enable enzyme catalyzed ligation.

Ligation products produced in accordance with ligase-dependent embodiments of the present invention may also be circularized and amplified by RCA.

The nucleic acid processing reactions of the present invention may include a variety of other reagents which may be included in the assays. These include reagents like salts, buffers, neutral proteins, e.g., albumin, detergents, etc., which may be used to facilitate optimal hybridization, strand synthesis and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target.

### 4. Oligonucleotides

The capture oligonucleotide of the present invention hybridizes to or “captures” the signaling oligonucleotide or the chimeric oligonucleotide depending on whether the target nucleic acid sequence is present in the test sample or not. In some embodiments, the capture oligonucleotide is immobilized on a solid surface whilst in others, it is free in solution.

In embodiments in which the capture oligonucleotide is immobilized on a solid surface, the surface can be comprised of natural, synthetic or naturally occurring materials that are synthetically modified including, but not limited to, cellulose materials such as paper, cellulose and cellulose derivatives such as cellulose acetate and nitrocellulose; glass or glass fibres; natural or synthetic cloth; plastics; nylon; porous gels such as agarose, silica gel, dextran and gelatin; porous fibrous matrixes; starch based materials such as Sephadex cross-linked dextran chains; ceramic materials; latex; films of polyvinyl chloride and polyamide; polystyrene; polycarbonate; and combinations of polyvinyl chloride-silica and the like.

In some embodiments, the solid surface forms a surface of a reaction vessel in which the processing reaction is performed. In other embodiments, the solid surface may also be the surface of a diagnostic strip that is inserted into the reaction vessel and removed following completion of the processing reaction for signal detection. In still other embodiments, the solid surface is a surface of a microparticle or bead, which is optionally tagged to facilitate identification. In other embodiments, the surface is a semiconducting nanowire, which is suitably configured as a field effect transistor, and which changes conductance upon binding or hybridization of a nucleic acid sequence (e.g., a capturable sequence) to a capture oligonucleotide immobilized to the nanowire surface (see for example, Cui et al., 2001, Science 293: 1289-1292; Hahm and Lieber, 2004, Nano Lett. 4: 51-54; Chen et al., 2003, Proc. Natl. Acad. Sci. USA 100: 4984-4989; Chen et al., 2004, J. Am. Chem. Soc. 126: 1563-1568 and Patolsky et al., 2004, Proc. Natl. Acad. Sci. USA 101: 14017-14022).

The capture oligonucleotides may be immobilized on a solid surface using any suitable technique. For example, Holstrom et al. (1993, Anal. Biochem. 209: 278-283) exploit the affinity of biotin for avidin and streptavidin, and immobilize biotinylated nucleic acid molecules to avidin/streptavidin coated supports. Another method which may be employed involves precoating of polystyrene or glass solid phases with poly-L-Lys or poly-L-Lys, Phe, followed by covalent attachment of either amino- or sulfhydryl-modified oligonucleotides using bifunctional cross linking reagents (Running et al., 1990, Biotechniques 8: 276-277; Newton et al., 1993, Nucleic Acids Res. 21: 1155-1162). Kawai et al. (1993, Anal. Biochem. 209: 63-69) describe an alternative method in which short oligonucleotide probes are ligated to form multimers before cloning thereof into a phagemid vector. The oligonucleotides are then immobilized onto a polystyrene plate and fixed by UV irradiation at 254 nm. Reference also may be made to a method for the direct covalent attachment of short, 5′-phosphorylated oligonucleotide primers to chemically modified polystyrene plates (Covalink™ plate, Nunc) (Rasmussen et al., 1991, Anal. Biochem. 198: 138-142). Regard may also be had to an article by O'Connell-Maloney et al. (1996, TIBTECH 14: 401-407) which discloses immobilization of biotinylated oligonucleotides and sulfhydrylated oligonucleotides respectively to a streptavidin-coated silicon wafer and an iodoacetamide-coated silicon wafer. Also, amino-modified oligonucleotides have been immobilized on isothiocyanate-coated glass (Guo et al., 1994, Nucleic Acids Res. 22: 5456-5465) and silane-epoxide-coated wafer (Eggers et al., 1994, BioTechniques 17: 516-5240). The aforementioned methods refer to post-synthetic attachment of oligonucleotide primers to a substrate. Alternatively, the oligonucleotide primers may be synthesized in situ utilizing, for example, the method of Maskos and Southern (1992, Nucleic Acids Res. 20 1679-1684) or that of Fodor et al. (supra).

Persons of skill in the art will recognize that the capture oligonucleotide can be immobilized on the solid surface either directly or indirectly. For example, a capture oligonucleotide may be adsorbed to a solid surface or alternatively covalently bound to a spacer molecule, which has been covalently bound to a solid surface. The spacer molecule may include a latex microparticle, a protein such as bovine serum albumin (BSA) or a polymer such as dextran or poly-(ethylene glycol). Alternatively, the spacer molecule may comprise a homo-polynucleotide tail such as, for example, oligo-dT.

The capture oligonucleotide may be arranged on the solid surface in any arrangement that facilitates detection of the signaling oligonucleotide in the event the signaling oligonucleotide is captured. In certain embodiments, the capture oligonucleotides are arranged on a solid surface in the form of a capture oligonucleotide array. The detection of the results of multiplexed reactions can be facilitated by immobilizing capture oligonucleotides corresponding to a particular target in a specific location on the array. In embodiments where the capture oligonucleotides are immobilized on microparticles or beads, detection of the results of multiplexed reactions could be facilitated by immobilizing capture oligonucleotides corresponding to a particular target on specific proportions of the total number of microparticles or beads used in the reaction.

Typically, capture oligonucleotides in accordance with the present invention are not target specific, but rather specific to individual (preferably) artificial capturable sequences contained within a chimeric oligonucleotide or a signaling oligonucleotide as defined herein, and permit their use as “universal arrays.” That is, arrays (either solid phase or liquid phase arrays) that contain the same or a finite set of capture oligonucleotides. By “liquid phase arrays” is meant an array in solution for analysis, for example, by flow cytometry. In some embodiments, the capture oligonucleotides are in the form of a universal surface; that is, a standard array, comprising a finite set of capture oligonucleotides that can be made and used in any application. Such universal arrays are used with substantially complementary signaling oligonucleotides, which bind to the capture oligonucleotides in the presence of target sequence. The end-user can customize the array merely by designing different chimeric oligonucleotides comprising any desired targeting sequence, which, as will be appreciated by those in the art, is generally simpler and less costly. In some embodiments, an array of different and usually artificial capture oligonucleotides is made; that is, the capture oligonucleotides do not have complementarity to known target sequences. A capturable sequence that is substantially complementary to a capture oligonucleotide of the array can then be incorporated into a chimeric oligonucleotide.

The signaling oligonucleotide of the present invention hybridizes to the capture oligonucleotide when the target nucleic acid sequence is present in the test sample and provides at least in part a detectable signal indicating the presence of the target nucleic acid sequence in the test sample. As such, the signaling oligonucleotide comprises a sequence of nucleotides, which can hybridize with the capture oligonucleotide and a signaling reagent that is associated with the oligonucleotide.

The signaling oligonucleotide may have a signaling reagent associated therewith which includes the following: (1) direct attachment of the signaling reagent to the signaling oligonucleotide; (2) indirect attachment of the signaling reagent to the signaling oligonucleotide; (i.e., attachment of the signaling reagent to a secondary intermediate which subsequently binds to the signaling oligonucleotide); and (3) attachment to a subsequent reaction product of the signaling oligonucleotide. Suitably, the signaling reagent is attached directly to the signaling oligonucleotide. The signaling reagent may be selected from a chromogen, a catalyst, an enzyme, a fluorophore, a luminescent molecule, a chemiluminescent molecule, a lanthanide ion such as Europium (Eu³⁴), a radioisotope and a direct visual signaling reagent. In the case of a direct visual signaling reagent, use may be made of a colloidal metallic or non-metallic particle, a dye particle, an enzyme or a substrate, an organic polymer, a latex particle, a liposome, dendrimer (or dendrimer-like or concatenated nucleic acid structures such as SAT) or other vesicle containing a signal producing substance and the like. A large number of enzymes suitable for use as signaling reagents is disclosed in U.S. Pat. No. 4,366,241, U.S. Pat. No. 4,843,000, and U.S. Pat. No. 4,849,338. Suitable enzyme signaling reagents useful in the present invention include alkaline phosphatase, horseradish peroxidase, luciferase, β-galactosidase, glucose oxidase, lysozyme, malate dehydrogenase and the like. The enzyme-signaling reagent may be used alone or in combination with a second enzyme that is in solution. Alternatively, a fluorophore, which may be used as a suitable signaling reagent in accordance with the present invention, includes, but is not limited to, fluorescein, rhodamine, Texas red, lucifer yellow or R-phycoerythrin. It will also be appreciated that, in the case of indirect attachment of the signaling reagent (2), reagents such as biotin, digoxigenin, streptavidin and various protein antigens act as a link and require the presence of a second intermediate for production of a detectable signal. For biotin, the secondary intermediate may include streptavidin enzyme conjugates. For antigen signaling reagents, secondary intermediates may include antibody-enzyme conjugates.

In certain embodiments in which the capture oligonucleotide is free in solution, the capture oligonucleotide comprises one part of a signal detection system and the signaling oligonucleotide comprises the other part of the signal detection system, wherein the individual parts cooperate to provide a first signal that indicates the presence of a target nucleic acid sequence or a second signal that indicates the absence of the target sequence in the test sample. In illustrative examples, the capture oligonucleotide comprises an acceptor fluorophore and the signaling oligonucleotide comprises a donor fluorophore such that when the signaling oligonucleotide hybridizes to the capture oligonucleotide, the acceptor and donor fluorphores are in sufficiently close proximity to induce fluorescence resonance energy transfer (FRET). The detection of FRET will signal the presence of the target nucleic acid sequence in the test sample, while the absence of FRET will signal the absence of that sequence in the test sample. As will be known to the skilled artisan, FRET can be detected in at least one of two ways: fluorescence or quenching. In fluorescence, a fluorescence detector is set to the emission spectra of the acceptor fluorophore and binding of the signaling oligonucleotide to the capture oligonucleotide is indicated by energy transfer from the donor to the acceptor and fluorescence from the acceptor. In quenching, the detector is set to the emission spectra of the donor fluorophore and binding of the signaling oligonucleotide to the capture oligonucleotide is indicated by energy transfer from the donor to the acceptor and quenching of emission from the donor.

The chimeric oligonucleotide of the present invention comprises a first targeting sequence that hybridizes to a subsequence of the target nucleic acid sequence and a capturable sequence that hybridizes to a sequence selected from the capture oligonucleotide or the signaling oligonucleotide. As described above, in the absence of a target nucleic acid sequence, the capturable sequence of the chimeric oligonucleotide hybridizes to the capture oligonucleotide and blocks signaling. In some embodiments, the capturable sequence of the chimeric oligonucleotide hybridizes preferentially to the capture oligonucleotide. This may be a result of the chimeric oligonucleotide being present in relatively higher concentrations than the signaling oligonucleotide or the chimeric oligonucleotide may be modified such that it hybridizes to the capture oligonucleotide with higher affinity than the signaling oligonucleotide.

Typically, the capturable sequence is a nucleic acid that is generally not native to the target sequence, i.e. is exogenous, but is added or attached to the targeting sequence. It should be noted that in this context, the “target sequence” can include the primary sample target sequence, or can be a derivative target such as a reactant or product of the reactions outlined herein; thus for example, the target sequence can be a PCR product, a first ligation probe or a ligated probe in an OLA reaction, etc. Capturable sequence serve as unique identifiers of a chimeric oligonucleotide and thus of the target sequence. In general, sets of capturable sequences and the corresponding capture oligonucleotides on, for example, arrays are developed to minimize cross-hybridization with both each other and other components of the reaction mixtures, including the target sequences and sequences on the larger nucleic acid sequences outside of the target sequences (e.g., to sequences within genomic DNA). Some capturable or “adapter” sequences are outlined in U.S. Appl. Pub. No. 20030096239. Exemplary capturable sequences are those that meet the following criteria. They are not found in a genome, suitably a human genome, and they do not have undesirable structures, such as hairpin loops.

In addition, as will be appreciated by those in the art, the capturable sequence can be incorporated into the chimeric oligonucleotide either on the 3′ or 5′ ends, or in an internal position, depending on the configuration of the system.

As will be appreciated by those in the art, the length of the capturable sequences will vary, depending on the desired “strength” of binding and the number of different capturable sequences desired. In some embodiments, capturable sequences generally range from about 6 to about 500 base pairs in length, usually from about 8 to about 100, and more usually from about 10 to about 25.

In some embodiments, the capturable sequence uniquely identifies the target sequence to which the targeting sequence binds. That is, while the capturable sequence need not bind itself to the target sequence, the system allows for identification of the target sequence by detecting the presence of the capturable sequence. Accordingly, detection of the capturable sequence then serves as an indication of the presence of the target sequence.

Methods and algorithms for identifying a suitable subsequence of the target nucleic acid sequence are well known to those experienced in the fields of molecular biology and are widely used. The methods include sequence alignment methods (Gotoh O. Multiple sequence alignment: algorithms and applications. Adv Biophys. 1999; 36:159-206. Lecompte O, Thompson J D, Plewniak F, Thierry J, Poch O./Multiple alignment of complete sequences (MACS) in the post-genomic era. Gene. 2001 May 30; 270(1-2):17-30.), the dotmatrix method and database searching methods such as the Basic Local Alignment Sequence Tool and the FASTA programs (Pearson W R. Flexible sequence similarity searching with the FASTA3 program package. Methods Mol. Biol. 2000; 132:185-219; Altschul, S. F., Gish, W., Miller, W., Myers, E. W. & Lipman, D. J. (1990) “Basic local alignment search tool.” J. Mol. Biol. 215:403-410). A subsequence of the target nucleic acid sequence may be a region that is conserved in a family of nucleic acid sequences or it may be a region that is unique to the particular target nucleic acid sequence.

Methods for designing targeting sequences that hybridize to subsequences of target nucleic acid sequences are also well known and widely used. In this regard, reference can be made to Dieffenbach et al., 1995 PCR Primer: A Laboratory Manual, CSHL press, Cold Spring Harbor, USA; Erlich, 1989, PCR Technology: Principles and Applications for DNA Amplification, Stockton Press, New York; Innis et al., 1990, PCR Protocols: A Guide to Methods and Applications, Academic Press, New York; Ellingboe et al., 1992, The PCR Technique: DNA Sequencing. Eaton Publishing Co., Natick, Mass.; Bej et al, 1991, Critical Reviews in Biochemistry and Molecular Biology 26: 301-334; Hughes et al., 2001, Nat. Biotechnol. 19: 342-347; Kane et al., 2000, Nucleic Acids Res. 28: 4552-4557; Relogio et al., 2002, Nucleic Acids Res. 30: e51; Wang et al., 2003, Bioinformatics 19: 796-802; Chen et al., 2002, BMC Bioinformatics 3: 27; Hill et al., 2002, Proceedings of Objects in Bio- & Chem-Informatics, OMG Object Management Group, Washington, D.C. (USA), pp 39-44; Li et al., 2001, Bioinformatics 17: 1067-1076; Lipshutz et al., 1999, Nat. Genet. 21: 20-24; Nielsen et al., 2003, Nucleic Acids Res. 31: 3491-3496; Raddatz et al., 2001, Bioinformatics 17: 98-99; Rahmann, 2003, Journal of Bioinformatics and Computational Biology 1: 343-361; Reymond et al., 2004, Bioinformatics 20: 271-273; Rimour et al., 2003, Proceedings of the first Healthgrid Conference, Lyon, France, pp. 88-96; Rouillard et al., 2002, Bioinformatics 18: 486-487; Rouillard et al., 2003, Nucleic Acids Res, 31: 3057-3062.

In general, targeting sequences are substantially complementary to subsequences of target nucleic acid sequences. For example, if a subsequence of target nucleic acid sequence is A-G-T-A-C-T-G, the complementary targeting sequence would be generated T-C-A-T-G-A-C. The properties of the subsequences of target nucleic acid sequences and their complements are calculated and used to optimise the design of targeting sequences. The properties that are considered include, but are not limited to, oligonucleotide length (in base residues), Tₘ, and propensity for self-hybridization. Algorithms for estimating the properties are well known to those skilled in the art and are widely used. Reference can be made to Jarman, 2004, Bioinformatics 20(10): 1644-1645; Gibbs et al., 1997, J. Virol. Methods 63(1-2): 9-16; Gibbs et al., 1998, J. Virol. Methods, 74(1): 67-76; Antoniw, 1995, Mol. Biotechnol. 4(2): 111-119; Le Guyader et al., 1996, Arch. Virol. 141(11): 2225-2235; Chen et al., 1995, Virus Res. 39(2-3): 365-375. Wilson et al., 1993, J. Clin. Microbiol. 323: 1573-1580; Chen et al., 1989, FEMS Microbiol. Lett. 57: 19-24; Greigen et al., 1994, J. Clin. Microbiol. 32: 335-351; Evertsson et al., 2000, APMIS 108: 385-392; Hryniewiecki et al., 2002, J. Heart Valve Dis. 11: 870-874; Rothman et al., 2002, J. Infect. Dis. 186: 1677-1681; McCabe et al., 1995, Pediatrics 95: 165-169; Lisby et al., 2002, Infect. Dis. Clin. North Am. 16: 393-412; Ley, 1998, Eur. J. Clin. Microbiol. Infect. Dis. 17: 247-253.

A cooperating oligonucleotide of the present invention comprises a second targeting sequence that hybridizes to a sequence selected from a different subsequence of the target nucleic acid sequence than the subsequence to which the first targeting sequence hybridizes; or a subsequence of a complementary strand of the target nucleic acid sequence, or at least one chimeric oligonucleotide. The cooperating oligonucleotide cooperates with the chimeric oligonucleotide, in a nucleic acid processing reaction and in the presence of the target nucleic acid sequence, to form a reaction product. In the context of the present invention, the reaction product comprises a first strand comprising at least one chimeric oligonucleotide as well as a second strand comprising at least one cooperating oligonucleotide, or extension product thereof, that blocks the hybridization of the capturable sequence of the chimeric oligonucleotide to the capture oligonucleotide, thereby allowing the signaling oligonucleotide to hybridize to the capture oligonucleotide.

Oligonucleotides for use in accordance with the present invention may be generated using any suitable method, such as, for example, the phosphotriester method as described in an article by Narang et al. (1979, Methods Enzymol. 68: 90) and U.S. Pat. No. 4,356,270. Alternatively, the phosphodiester method as described in Brown et al. (1979, Methods Enzymol. 68: 109) may be used for such preparation. Automated embodiments of the above methods may also be used. For example, in one such automated embodiment, diethylphosphoramidites are used as starting materials and may be synthesised as described by Beaucage et al., (1981, Tetrahedron Letters 22: 1859-1862). Reference also may be made to U.S. Pat. Nos. 4,458,066 and 4,500,707, which refer to methods for synthesising oligonucleotides on a modified solid support. It is also possible to use an oligonucleotide which has been isolated from a biological source (such as a denatured strand of a restriction endonuclease digest of plasmid or phage DNA). In some embodiments, the oligonucleotides are synthesised according to the method disclosed in U.S. Pat. No. 5,424,186 (Fodor et al.). This method uses lithographic techniques to synthesise a plurality of different oligonucleotides at precisely known locations on a substrate surface. Alternatively, oligonucleotides may be generated using PCR-based methods such as those described by Antson et al., 2000, Nucleic Acid Research 28(12): e58. These PCR-based methods are particularly suitable for generating longer oligonucleotides, of typically more than 100 nucleotides.

Nucleic acid hybridizations in accordance with the method of the present invention are performed under suitable conditions for hybridizing oligonucleotides to each other, or, to target nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridization takes place is influenced by the length of the nucleic acids, the pH, the temperature, the concentration of mono- and divalent cations, the proportion of G and C nucleotides in the hybrid-forming region of nucleic acids, the viscosity of the medium and the possible presence of denaturants. Such variables also influence the time required for hybridization. The preferred conditions will therefore depend upon the particular application. Such empirical conditions, however, can be routinely determined without undue experimentation.

In certain embodiments, high discrimination hybridization conditions are used in the methods of the invention. For example, reference may be made to Wallace et al. (1979, Nucl. Acids Res. 6: 3543) who describe conditions that differentiate the hybridization of 11 to 17 base long oligonucleotide probes that match perfectly and are completely homologous to a target sequence as compared to similar oligonucleotide probes that contain a single internal base pair mismatch. Reference also may be made to Wood et al. (1985, Proc. Natl. Acid. Sci. USA 82: 1585) who describe conditions for hybridization of 11 to 20 base long oligonucleotides using 3M tetramethyl ammonium chloride wherein the melting point of the hybrid depends only on the length of the oligonucleotide probe, regardless of its GC content. In addition, Drmanac et al. (supra) describe hybridization conditions that allow stringent hybridization of 6-10 nucleotide long oligomers, and similar conditions may be obtained most readily by using nucleotide analogues such as ‘locked nucleic acids (Christensen et al., 2001 Biochem J354: 481-4).

Generally, a hybridization reaction can be performed in the presence of a hybridization buffer that optionally includes a hybridization-optimising agent, such as an isostabilizing agent, a denaturing agent and/or a renaturation accelerant. Examples of isostabilizing agents include, but are not restricted to, betaines and lower tetraalkyl ammonium salts. Denaturing agents are compositions that lower the melting temperature of double stranded nucleic acid molecules by interfering with hydrogen bonding between bases in a double stranded nucleic acid or the hydration of nucleic acid molecules. Denaturing agents include, but are not restricted to, formamide, formaldehyde, dimethylsulphoxide, tetraethyl acetate, urea, guanidium isothiocyanate, glycerol and chaotropic salts. Hybridization accelerants include heterogeneous nuclear ribonucleoprotein (hnRP) A1 and cationic detergents such as cetyltrimethylammonium bromide (CTAB) and dodecyl trimethylammonium bromide (DTAB), polylysine, spermine, spermidine, single stranded binding protein (SSB), phage T4 gene 32 protein and a mixture of ammonium acetate and ethanol.

It will also be appreciated that if the nucleotide sequences of the present invention contain two strands (e.g., genomic DNA) or have secondary structure which may preclude oligonucleotide hybridization and/or extension (e.g., RNA), it is desirable to separate the strands of the nucleic acid sequence, either as a separate step or simultaneously with the synthesis of the extended primer molecule. This strand separation can be accomplished by any suitable denaturing method including physical, chemical or enzymatic means. One physical method of separating the strands of the polynucleotide sequence involves heating the nucleic acid sequence until it is substantially completely (>99%) denatured. Typical heat denaturation may involve temperatures ranging from about 80° C. to 105° C. for times ranging from about 1 to 10 minutes. Strand separation may also be induced by an enzyme from the class of enzymes known as helicases or the enzyme RecA, which has helicase activity and in the presence of riboATP (rATP) is known to denature DNA. Suitable reaction conditions for separating the strands of nucleic acids with helicases are described by Kuhn Hoffmann-Berling (1978, CSH-Quantitative Biology 43 63), and techniques for using RecA are reviewed in Radding (1982, Ann. Rev. Genetics 16 405-437). Alternatively, electrical denaturation may be used to effect denaturation of the polynucleotide sequence by, for example, applying low voltage electricity through the test sample (Purvis et al., 1996, 4th World Congress on Biosensors, Bangkok. p 39, Elsevier Advanced Technology, Oxford, which is hereby incorporated by reference), or by treating the test sample with dilute acid (e.g., 0.25 M HCl for 7.5 to 10 minutes (Meinkoth and Wahl, 1984, Anal. Biochem. 138 267-284).

### 5. Detection

Depending on the nature of the signaling reagent, detecting the detectable signal from the signaling oligonucleotide of the present invention may be carried out by visual inspection or by an instrumental means. A signal may be instrumentally detected by irradiating a fluorescent label with light and detecting fluorescence in a fluorimeter; by providing for an enzyme system to produce a dye which could be detected using a spectrophotometer; or detection of a dye particle or a coloured colloidal metallic or non metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focussed beam or laser light. In such a case, a charge couple device (CCD) or a photocell can be used to scan for emission of light from a capture oligonucleotide/signaling oligonucleotide hybrid from each location in an array and record the data directly in a digital computer. Alternatively, if the capture oligonucleotides are immobilized on microparticles or beads, the signaling reagent may be detected using fluorescence activated cell sorting (FACS) technology. In some cases, instrumental detection of the signal may not be necessary. For example, with colloidal metallic particles or enzymatically generated colour spots associated with the array format, as herein described, visual examination of the array will allow interpretation of the pattern on the array.

In some embodiments, the solid surface onto which the capture oligonucleotides are immobilized may also be associated with pattern recognition apparatus and software to convert the pattern of signals from, for example the array, into a plain language genetic profile. In certain embodiments, the detection of a signal generated from a signaling reagent on the array is performed using a ‘chip reader’. A detection system that can be used by a ‘chip reader’ is described for example by Pirrung et al (U.S. Pat. No. 5,143,854). The chip reader will typically also incorporate some signal processing to determine whether the signal at a particular array position or feature is a true positive or maybe a spurious signal. Exemplary chip readers are described for example by Fodor et al (U.S. Pat. No. 5,925,525).

In specific embodiments, the detectable signal of the present invention not only indicates the presence of the target nucleic acid sequence, but also indicates the amount of target nucleic acid sequence, depending on the amount of the signaling oligonucleotide which has hybridized to the capture oligonucleotide. In this regard, quantification of the target nucleic acid sequence may be performed by any method known to those skilled in the art such as, but not limited to, via reference to a standard nucleic acid sample having a known concentration.

Interpretation of the detectable signals of the present invention may also be aided by the inclusion of positive and negative controls. Persons skilled in the art could readily ascertain suitable controls. A positive control may include a chimeric oligonucleotide having a first subsequence that is specific for a sequence that would be present in the test sample, e.g., β-actin sequences. A negative control may include a chimeric oligonucleotide that lacks a first target specific subsequence or that has a target specific subsequence that does not substantially hybridize to the target nucleic acid sequence.

### 6. Test Samples

Suitable test samples, which may be used in accordance with the present invention, include extracts of single or double stranded nucleic acid, or copies thereof, obtained from any organism. Extracts may be obtained from any source from the organism, such as but not limited to, lysates, cells, tissues or other materials derived from viruses, fungi, bacteria, plants and animals.

Sample extracts of nucleic acid, such as DNA or RNA, may be prepared following a cell lysis step which includes, but is not limited to, lysis effected by treatment with SDS, osmotic shock, sonication, guanidinium isothiocyanate and lysozyme. Suitable DNA, which may be used in accordance with the present invention, includes genomic DNA or cDNA. Such DNA may be prepared by any one of a number of commonly used protocols as for example described in CURRENT PROTOCOLS IN MOLECULAR BIOLOGY (Ausubel, et al., eds.) (John Wiley & Sons, Inc. 1995) and MOLECULAR CLONING. A LABORATORY MANUAL (Sambrook, et al., eds.) (Cold Spring Harbor Press, 1989). Sample extracts of RNA may be prepared by any suitable protocol as for example described in CURRENT PROTOCOLS IN MOLECULAR BIOLOGY (supra), MOLECULAR CLONING. A LABORATORY MANUAL (supra) and Chomczynski and Sacchi (1987, Anal. Biochem. 162: 156).

As will be appreciated by those in the art, the sample extract may comprise any number of things, including, but not limited to, bodily fluids (including, but not limited to, blood, urine, serum, lymph, saliva, anal and vaginal secretions, perspiration and semen, of virtually any organism); environmental samples (including, but not limited to, air, agricultural, water and soil samples); biological warfare agent samples; research samples; purified samples, such as purified genomic DNA, RNA, proteins, etc.; raw samples (bacteria, virus, genomic DNA, etc.). A sample also can comprise a combination of samples, such as two or more samples from different sources mixed together. As will be appreciated by those in the art, virtually any experimental manipulation may have been done on the sample.

### 7. Reaction Formats

The analytical methods of the present invention may be carried out in any suitable reaction vessel including tubes, microwells, wafers (i.e., chips) and microfluidic devices. In some embodiments, the vessels are manufactured or prefabricated with at least one of a capture oligonucleotide, a chimeric oligonucleotide, a cooperating oligonucleotide, a signaling oligonucleotide, reaction buffers, enzymes, signaling reagents and nucleotide precursors. Suitably, the reaction components are included in the reaction vessel or vessels either in solution or lyophilized form in amounts that are preoptimized for conducting an analytical method of the present invention. In illustrative examples of this type, the end user simply adds to such reaction vessels at least one of a nucleic acid sample, a nucleic acid processing enzyme, a chimeric oligonucleotide and a cooperating oligonucleotide, to conduct the assay.

In some embodiments, the reaction vessel is placed in a thermocontrollable environment, e.g., a thermal cycler, incubator etc to perform various steps of the analytical method of the present invention. In other embodiments, the reaction vessel is capable of performing one or more operations for use in these analytical methods. These operations include but are not limited to: mixing; filtration; nucleic acid extraction; nucleic acid purification; binding; elution; thermal control for hybridization, for conducting nucleic acid processing reactions (e.g., PCT, LCR, OLA, RCR etc) and for denaturation of nucleic acid hybrids; and detection of the signaling oligonucleotide. Illustrative devices of this type are disclosed for example in U.S. Pat. Appl. Pub. Nos. 2002/0115200, 2002/0173032, 2003/0008286 and 2005/0142565.

### 8. Kits

All the essential components required for analysing a target nucleic acid sequence in a test sample according to the method of the present invention may be assembled together in a kit. The kits may optionally include appropriate components for visualising the signaling reagent, positive and negative controls, dilution buffers and the like. Also included may be components suitable for subjecting nucleic acid to a nucleic acid processing reaction. These components include various polymerases such as, but not limited to, Taq polymerase, reverse transcriptase, DNA ligase etc. (depending on the nucleic acid processing reaction technique employed), nucleotide precursors and buffer solutions. Such kits may also comprise distinct containers for the individual components. In some embodiments, the kits comprise instructions for performing the methods of the present invention.

In specific embodiments, the kit of the present invention comprises a reaction vessel having capture oligonucleotides immobilized thereto and containing a pre-prepared mixture of reagents, suitably in lyophilized form, as described for example in Section 7.

In order that the invention may be readily understood and put into practical effect, particular preferred embodiments will now be described by way of the following non-limiting examples.

### EXAMPLES

### Example 1

### RT-PCR Detection of the Influenza a Polymerase Gene Segment PB2

### Primer Design

RT-PCR primers are designed to amplify a part of the PB2 segment of influenza A virus. Inosine or mixed bases are used at a number of positions so that the polymerase gene from a number of different viruses can be amplified. One of the primers was chimeric in so far as the 5′ end of the PCR primer contains a capturable sequence or “tag”. The capturable sequence used in this example is 5′-CTT TAA TCT CAA TCA ATA CAA ATC-3′ ([SEQ ID NO: 1], labelled as B′ in FIG. 2) and is one of the sequences whose design is described by U.S. Pat. No. 6,027,884. Any other capturable sequence that is not found in the target sequence (influenza A virus) may be used.

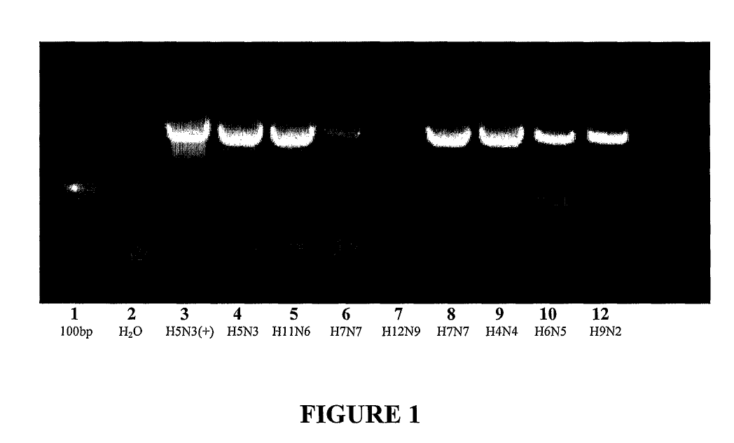

The chimeric influenza forward primer Ch-PB2(1)F comprised the sequence 5′-CTT TAA TCT CAA TCA ATA CAA ATC AG(C/T) TCI TC(C/T) TT(C/T) AG(C/T) TT(C/T) GG-3′ [SEQ ID NO: 2]. The influenza reverse primer PB2(2)R comprised the sequence 5′-AGT AT(T/C) CTC AT(T/C)CC(T/A) GAN CC-3′ [SEQ ID NO: 3]. The expected size of the product generated from these primers is approximately 1010 bp. However, this may vary depending upon which virus is present in a sample of interest as the length of the PB2 coding sequence may vary slightly between isolates (FIG. 1).

### Extraction of RNA from Sample

Viral RNA was extracted from the sample (e.g., amniotic fluid or clinical sample) using a QIAGEN RNA easy extraction kit by following the manufacture's instructions. 100 μL of sample was inactivated by addition of 600 μL of a guanidium denaturant and 6 μL of 2-mercaptoethanol prior to use in the QIAGEN extraction protocol. The extracted RNA was resuspended in 50 μL of Rnase free water (QIAGEN). Two μL of the resuspended RNA was used in the RT-PCR reaction.

### Quantification

Approximately 100 picomoles of capture oligonucleotide (labelled as B in FIG. 2) (spacer-5′-GAT TTG TAT TGA TTG AGA TTA AAG-3′; [SEQ ID NO: 4]) are bound onto the wells of a 96 well plate, or strips of tubes that are of a shape suitable for use in a thermal cycling machine. The binding of the capture oligonucleotide to the substratum may be achieved by using a 5′ modifying group (e.g., amino group with a (CH2)6 “spacer” sequence (or similar) between the amino group and the 5′ end of the capture oligonucleotide). Alternatively, a 5′ biotin moiety which can bind non-covalently but with very high affinity to plates coated with Streptavidin is incorporated at one end of the capture oligonucleotide.

A signaling oligonucleotide is designed that contains the sequence 5′-CTT TAA TCT CAA TCA ATA CAA ATC-3′ ([SEQ ID NO: 1], labelled as B″ in FIG. 2) conjugated to a detection moiety (e.g., a latex microbead) is added to each well of a microwell plate that has been precoated with the capture oligonucleotide. The signaling oligonucleotide is added at an approximately equal mass to the mass of capture oligonucleotide that has been immobilized. The exact mass of capture oligonucleotide to be added should be empirically determined by titration for each assay and each batch of reagents.

### Standards for Quantification

A 986 bp segment of the PB2 gene has been cloned into the plasmid PCR TOPO2.1 (Invitrogen). This plasmid (referred to herein as the “control plasmid”) has sequences complementary to the virus sequence complementary part of the forward primer Ch-PB2(1)F and the reverse primer PB2(2)R. The control plasmid can be used in serial dilution as a quantification standard in the assay and as a reagent control for the RT-PCR reaction. Serial dilutions of the plasmid in the range of 0 to 10,000 picomoles per well may be used as quantification and reagent controls. When approximately 100 picomoles of capture primer is immobilized per well, the concentration range for the quantification standards should be in the range zero to 10 nanomoles per well including 0, 3, 10, 30, 100, 300, 1000, 3000 and 10,000 picomoles per well, with duplicate wells for each concentration of standards.

### The RT-PCR Reaction

1. The thermal cycler was programmed so that cDNA synthesis is followed immediately by PCR amplification, as follows:

cDNA Synthesis:

1 cycle: 30 min at 46° C. plus 10 min at 60° C.

Denaturation:

1 cycle: 94° C. for 2 min

PCR Amplification:

8 cycles of step down PCR consisting of:

15 s denaturation at 94° C.

30 s annealing at 56° C., 54° C., 52° C., 50° C., 48° C., 46° C., 44° C., 42° C. in each of the successive “step down cycles”

75 s extension at 68° C.
followed by:
36 cycles: 94° C. for 15 s (denature)
40° C. for 30 s (anneal)
68° C. for 75 s (extend)

Final Extension (Optional):

1 cycle: 68° C. for 5 min

2. Add the following to a 0.2 mL, nuclease-free, thin-walled PCR tube (that has the capture oligonucleotide pre-bound to its walls) on ice:

2×Reaction mix 25 μL

Template RNA 2 μL or serial dilutions of the control plasmid (as quantification standards) (2 μL)

Sense primer Ch-PB2(1)F 2 μL (160 pmoles)

Anti-sense primer 2 μL (160 pmoles)

SuperScript III RT/PlatinumTaq Mix (In Vitrogen) 2 μL

2 μL of signaling oligonucleotide (100 pmoles or other amount predetermined by titration)

RNAse free water to 50 μL

3. Gently mix and centrifuge briefly to ensure that all the components are at the bottom of the amplification tube. Place the reaction plate or tubes in the preheated thermal cycler programmed as described above.

4. As the reaction proceeds, the chimeric influenza forward primer Ch-PB2(1)F 5′-CTT TAA TCT CAA TCA ATA CAA ATC AG(C/T) TCI TC(C/T) TT(C/T) AG(C/T) TT(C/T) GG-3′ [SEQ ID NO: 2] is incorporated into a double stranded PCR product and is sequestered such that it cannot compete for binding between the immobilized capture oligonucleotide (spacer-5′-GAT TTG TAT TGA TTG AGA TTA AAG-3′; [SEQ ID NO. 4]) and the signaling oligonucleotide.

Thus the amount of bound signaling oligonucleotide at the “annealing” stage of each PCR cycle increases as the reaction proceeds.

The intensity of the signal in the tubes/wells containing unknowns is compared with the intensity of the signal in the tubes/wells that were spiked with serially diluted plasmid standards in order to produce a standard curve. By comparison with the standard curve, the concentration of the target virus sequence in the sample can be calculated.

It is not obligatory to use a separate series of tubes/wells to generate a standard curve. In the latter case an “internal standard” can be used in which a known amount of a sequence unrelated standard RNA or plasmid can be co-amplified in the same reaction tube as the unknown sample using a different chimeric primer and signaling oligonucleotide pair that detects only the standard RNA or plasmid. The amount of signal generated by the unknown sample can then be compared to the signal generated by the internal standard RNA or plasmid in the same tube.

Measurement of the intensity of the signal could be done at the end of all cycles of the PCR reaction or could be checked by a reading device at each cycle of the PCR reaction.

In another embodiment, the signaling oligonucleotide may alternatively be pre-incorporated (e.g., by freeze drying) into the reaction tube, along with all ingredients other than the template RNA. The reaction mix can be reconstituted by addition of water).

### Example 2

### “End Point” Detection

This example utilizes an “end-point” detection step that is separate from the amplification step. The detection reagents and signaling reagents are present in a 96 well plate with approximately 100 picomoles of capture oligonucleotide immobilized per well as described in Example 1. The capturable chimeric RT-PCR primer is as given in Example 1 (Ch-PB2(1)F 5′-CTT TAA TCT CAA TCA ATA CAA ATC AG(C/T)-TCI TC(C/T) TT(C/T) AG(C/T) TT(C/T) GG-3′) [SEQ ID NO: 2]. The signaling oligonucleotide is as in Example 1 but is biotinylated and is present in the detection well in approximately equimolar amount as the capture oligonucleotide (although this will be titrated for each different analyte). The RT-PCR primers are identical to those used in Example 1.

The RT-PCR reactions are carried out as in Example 1, but without the inclusion of capture oligonucleotide and signaling oligonucleotide during the RT-PCR. Serially diluted plasmid controls are included as quantification standards. Upon completion of the RT-PCR reaction the reaction product is then heated at 94° C. for 5 minutes then quenched in wet ice and transferred to the detection plate.

As the PCR reaction proceeds, the chimeric capturable PCR primer is incorporated into double stranded product and competes less effectively with the capture oligonucleotide for binding to the signaling oligonucleotide. The greater the amount of PCR product is present at the end of reaction, the less the competition for binding of the signaling oligonucleotide.

After 20 minutes at 37° C., the detection plate is washed three times in 300 μL per well of 1×SSC pH 7.2 then the Streptavidin Peroxidase conjugate (Roche Cat. No. 1089153) is added to the wells at a dilution of 1:10,000 in PBS/0.1% Tween/0.5% BSA, incubated at 37° C. for 30 minutes then washed three times in PBS/Tween. Plates are drained by inversion then Substrate is then added to each to each well. 0.42 mM TMB (Roche Cat. No. 11484281001), 0.004% H2O2 (v/v), in 100 mM sodium acetate/citric acid, pH 4.9 is then added. Stop the reaction with 2 M H₂SO₄. The formed product is at first blue and after stopping yellow and soluble in water. Absorbance is measured on an ELISA plate reader at 450 nm against reference wavelength 650 nm.

### Example 3

### Ligase-Dependent Reactions

The DNA used in this illustrative example is a control plasmid DNA containing a double stranded DNA copy of the coding region of the influenza A PB2 gene segment. In this plasmid a 986 bp segment of the PB2 gene has been inserted into the plasmid PCR TOPO2.1 (Invitrogen).

### Chimeric Ligation Oligonucleotide Sequences

The ligatable oligonucleotides are designed to be complementary to a region of influenza A that includes the codon 627 (or a position equivalent numeric position in the sequence of PB2 in influenza A). Ch-PB2U 5′-CTT TAA TCT CAA TCA ATA CAA ATC TTG CAG CIG CIC CAC CIG-3′ ([SEQ ID NO: 5], labelled B′-E1′ in FIG. 4). PB2D-5′-AIC AIA GIA GIA TGC AGT-3′ ([SEQ ID NO: 6], labelled E2′ in FIG. 4). The capturable sequence used in the upstream chimeric ligation oligonucleotide is the same as the sequence used in Example 1.

For exemplary purposes, the ligase dependent reaction is carried out using the standard plasmid DNA described above as the target, using a thermostable DNA ligase. The process to amplify and detect a part of the PB2 segment of influenza A in a control plasmid (described above) is carried out as follows (protocol modified from Belgrader et al., 1995, Genetic Identity Conference Proceedings, Sixth International Symposium on Human Identification):

A 5 μL aliquot of the plasmid is diluted in 20 μL of LCR mix containing 50 mM Tris/HCl pH 8.5, 50 mM KCl, 10 mM MgCl₂, 1 mM NAD⁺, 10 mM DTT, LCR oligonucleotide set (50 pmol of each primer, Ch-PB2U and PB2D) and 10 units of Taq DNA ligase. Thermal cycling is performed for 1 cycle of 95° C. for 2 min to denature, then 20 to 25 cycles of 95° C. for 30 sec to denature and 65° C. for 4 min to ligate.

The reaction vessel for the ligase mediated reaction steps incorporates a detection system involving a second, specially designed cooperating oligonucleotide (labelled as B2-E2 in FIG. 4).

For the detection system, approximately 50 picomoles of capture oligonucleotide (spacer-5′-GAT TTG TAT TGA TTG AGA TTA AAG-3′) ([SEQ ID NO: 4], labelled B in FIG. 4) are bound onto the wells of a 96 well plate, or strips of tubes that are of a shape suitable for use in a thermal cycling machine. The binding of the capture oligonucleotide to the well may be achieved by using a 5′ modifying group (e.g., amino group with a (CH₂)₆ “spacer” sequence (or similar) between the amino group and the 5′ end of the capture oligonucleotide). Alternatively a 5′ biotin moiety which can bind non-covalently but with very high affinity to plates coated with Streptavidin is incorporated at one end of the capture oligonucleotide. Note that the capture oligonucleotide in this ligase mediated embodiment has the same sequence as the capture oligonucleotide used in Example 1. The sequence is complementary to the non-influenza sequence complementary part of the chimeric ligation oligonucleotide ChPB2U.

A signaling oligonucleotide is designed that contains the sequence 5′-CTT TAA TCT CAA TCA ATA CAA ATC-3′ [SEQ ID NO: 1] conjugated to a detection moiety (e.g., a latex bead) is added to each well of a microwell plate that has been pre-coated with the capture oligonucleotide (spacer-5′-GAT TTG TAT TGA TTG AGA TTA AAG-3′) [SEQ ID NO: 4]. The signaling oligonucleotide is added at an approximately equal mass to the mass of capture oligonucleotide that has been immobilized. The exact mass of capture oligonucleotide to be added should be empirically determined by titration for each assay and each batch of reagents.

### The Ligase Mediated Reaction Comprises

1. Denaturing the target plasmid DNA at 95° C. for 2 min.

2. Hybridizing a first targeting sequence of a chimeric oligonucleotide (Ch-PB2U) to a first subsequence of the target nucleic acid sequence to form a first hybrid, wherein the 3′ nucleotide of the chimeric oligonucleotide is complementary to the 5′ nucleotide of the first subsequence.

3. Hybridizing the second targeting sequence of a first cooperating oligonucleotide (PB2D) to a second subsequence of the target nucleic acid sequence, wherein the second subsequence is located adjacent to the first subsequence, to form a second hybrid.

4. Ligating the chimeric oligonucleotide with the first cooperating oligonucleotide in the presence of the 5′ nucleotide of the first subsequence using the enzyme Taq DNA ligase to form a first duplex comprising the first and second subsequences of the target nucleic acid sequence and a ligation product that comprises both the chimeric oligonucleotide and the first cooperating oligonucleotide.

5. Denaturing the first duplex at 95° C. for 30 sec to free the ligation product from the target nucleic acid.

6. Hybridizing a second specially designed cooperating oligonucleotide (B2-E2 in FIG. 4) to the chimeric oligonucleotide and first cooperating sequence portions of the ligation product to form a reaction product. The second specially designed cooperating oligonucleotide may be added at the end of the ligase mediated cycling process or may be present in the reaction mix from the beginning of the process.

7. The reaction product formed above results in sequestration of a blocking oligonucleotide sequence that is contained within the second cooperating oligonucleotide (labelled B2-E2 in FIG. 4). The sequestration of the blocking sequence allows the signaling oligonucleotide to hybridize to the immobilized capture oligonucleotide and the resultant generation of a signal (by a local concentration of the latex microbead) that is quantitatively related to the amount of a target sequence.

The signal generated is compared with a standard curve generated by the ligase mediated detection process from serial dilutions of a known starting concentration of a control plasmid and the starting concentration of the unknown sample can be estimated.

### Example 4

### Rolling Circle Amplification Reactions

This example describes detection and quantification of a region of the PB2 gene segment of influenza including the codon 627. In this example, the plasmid PCR-TOPO2.1 (Invitrogen) with a 986 bp DNA insert of the PB2 gene segment is used as the target. The protocol for the initial ligation and RCA steps is described in U.S. Pat. No. 5,854,033.

1. Several serial dilutions of the plasmid ranging from 1 nanogram to 300 nanogram in each dilution are heat denatured in separate plastic tubes for 4 minutes at 97° C. and incubated under ligation conditions in the presence of an 5′ phosphorylated open circle probe whose 5′ and 3′ ends are identical to the target specific portions of the ligation oligonucleotides CH-PB2U and PB2D respectively. Open circle cooperating oligonucleotide of 95 nucleotides is used with the following sequence 5′-AIC AIA GIA GIA TGC AGT TCA TAA GAC TCG TCA TGT CTC AGC AGC TTC TAA CGG TCA CTA ATA CGA CTC ACT ATA GG TTG CAG CIG CIC CAC CIG-3′ [SEQ ID NO: 7]. T4 DNA ligase (New England Biolabs) is present in the reaction mix at a concentration of 5 units per μL in a buffer consisting of 10 mM Tris-Hcl (pH 7.5), 0.20M NaCl, 10 mM MgCl₂, 2 mM ATP. The concentration of the open circle probe is 80 nM. The total reaction volume is 40 microlitres. The ligation is carried out for 25 minutes at 37° C.

2. 25 μL are taken from each tube and mixed with an equal volume of a buffer consisting of 50 mM Tris-Hcl (pH 7.5), 10 mM MgCl₂, 1 mM DTT, 400 μM each of dTTP, dATP, dGTP, dCTP, which also contains a chimeric 42 base rolling circle replication primer with the following sequence, at a concentration of 0.2 μM, 5′-CTT TAA TCT CAA TCA ATA CAA ATC GCT GAG ACA TGA CGA GTC-3′ [SEQ ID NO: 8]. Note that a region of this sequence is a capturable sequence identical to the capturable sequence used in Example 1 for RT-PCR. A different region is complementary to the open circle cooperating oligonucleotide [SEQ ID NO: 7].

3. Φ29 DNA polymerase (160 ng per 50 μL) is added and the reaction mixture is incubated for 30 minutes at 30° C.

4. To the above reactions another cooperating oligonucleotide, i.e., reverse primer, is added that is complementary to the newly synthesised strands. The concentration of this primer is 0.2 μM and the sequence is 5′-AAT ACG ACT CAC TAT AGG-3′ [SEQ ID NO: 9]. The reaction is allowed to continue for another 30 minutes at 30° C. and a second strand is synthesised that is reverse complementary to the sequence that was initially complementary to the circularized cooperating oligonucleotide and will include a sequence (which acts as a blocking sequence) complementary to the a region of SEQ ID NO: 8.

5. Each reaction tube contains an immobilized capture oligonucleotide [SEQ ID NO: 4] that is complementary to a region of SEQ ID NO: 8 (above) and a signaling oligonucleotide identical to the signaling oligonucleotide used in Example 1. As the synthesis of the second strand proceeds in step 4, a blocking sequence is synthesized and this sequence sequesters a region of SEQ ID NO: 8, rendering it less available to compete for the binding between the immobilized capture oligonucleotide sequence [SEQ ID NO: 4] and the signaling oligonucleotide. As the amount of second strand product accumulates the amount of bound signaling oligonucleotide will increase. The amount of signal generated is proportional to the amount of plasmid added to the starting reaction.

6. In another example of RCA the procedure is identical to the previous example, except the detection step is done at the completion of the reaction and a 25 μL sample of each reaction is transferred to a separate detection well in a microwell plate. The detection wells are precoated with 100 picomoles of the capture oligonucleotide and contain the signaling oligonucleotide (100 picomoles) conjugated to biotin. The concentration of bound signaling oligonucleotide is then detected using streptavidin peroxidase (Roche Cat. No. 1089153) and the substrate TMB (using the same method as described above in Example 2).

### Example 5

### “End Point” Detection Ii

This example shows another embodiment of an end point detection step. The detection reagents and signaling reagents are present in a 96 well plate with approximately 0.01 picomoles of capture oligonucleotide immobilized per well as described in Example 1. The capturable chimeric RT-PCR primer is 5′-CTT TAA TCT CAA TCA ATA CAA ATC GAI GTI AGI GAI ACI CAI GG-3′ [SEQ ID NO: 10]. The signaling oligonucleotide is as in Example 1 but is FAM labeled and is present in the detection well in approximately equimolar amount as the capture oligonucleotide (although this will be titrated for each different analyte). The PCR primers are as listed below in the tables at [0233] (the PCR-TAG primer and the PB2(2) reverse primer).

The PCR reactions were carried out as in Example 1, but starting with 200 picograms of cDNA as the target and omitting the initial RT (reverse transcription) step. The reaction was carried out without the inclusion of capture oligonucleotide and signaling oligonucleotide during the PCR. Upon completion of the PCR reaction the reaction product is processed as described below in paragraphs [0233] to [0241].

As the PCR reaction proceeds, the chimeric capturable PCR primer (PCR-TAG primer) is incorporated into double stranded product and competes less effectively with the capture oligonucleotide for binding to the signaling oligonucleotide. The greater the amount of PCR product is present at the end of reaction, the less the competition for binding of the signaling oligonucleotide.

The specific reaction conditions for the detection of H7N7 influenza A cDNA are as follows:

| | |
|---|---|
| DNA template (H7N7 influenza A CDNA) | 200 pg |
| PCR-Tag primer: 5′-CTT TAA TCT CAA TCA ATA | 0.5 to 1 |
| CAA ATC GAI GTI AGI GAI ACI CAI GG-3′ | pmole |
| [SEQ ID NO: 10] | |
| PB2(2) reverse primer: 5′-AGT ATY CTC ATY | 4 pmole |
| CCW GAI CC-3′ | |
| [SEQ ID NO: 3] | |
| dNTPs | 0.2 mM |
| DNA polymerase | 1 unit |
| MgCl₂ | 2 mM |
| PCR buffer | 1x |
| Water to a final volume of | 50 μL |

After PCR amplification (35 cycles), the PCR product is diluted 1/2.

Prepare the following mixture:

| | |
|---|---|
| Anti-tag: (5′-biotin-GAT TTG TAT TGA | 0.01 pmole |
| TTG AGA TTA AAG-3′, | |
| [SEQ ID NO: 4]) | |
| Tag: (5′-FAM-CTT TAA TCT CAA TCA ATA | 0.25 pmole |
| CAA ATC-3′, | |
| [SEQ ID NO: 1]) | |
| *total volume of anti-tag and tag is | 50 μL |
| made up with water to | |
| PCR product | 50 μL |

Transfer 100 μL of reaction mixture to each well on the streptavidin plate and incubate at 37° C. 40 min.
Tip off the mixture from the plate and wash with PBS for 4 times.
Add 100 μL (15 m units) of peroxidase conjugated anti-FAM antibodies diluted 1:1,000 in antibody diluent (150 mM Tris-Hcl, 100 mM NaCl, 2% V/V FBS (fetal bovine serum)) to each well of streptavidin plate and incubate at 37° C. for 40 min.
Tip off the mixture from the plate and wash with PBS for 4 times.
Add 100 μL of BM blue (Roche) substrate to each well of streptavidin plate and incubate at room temperature for 5 min.
Add 100 μL of H₂SO₄ to each well of streptavidin plate and incubate at room temperature for 10 min.
Measure the absorbance at 450 nm.

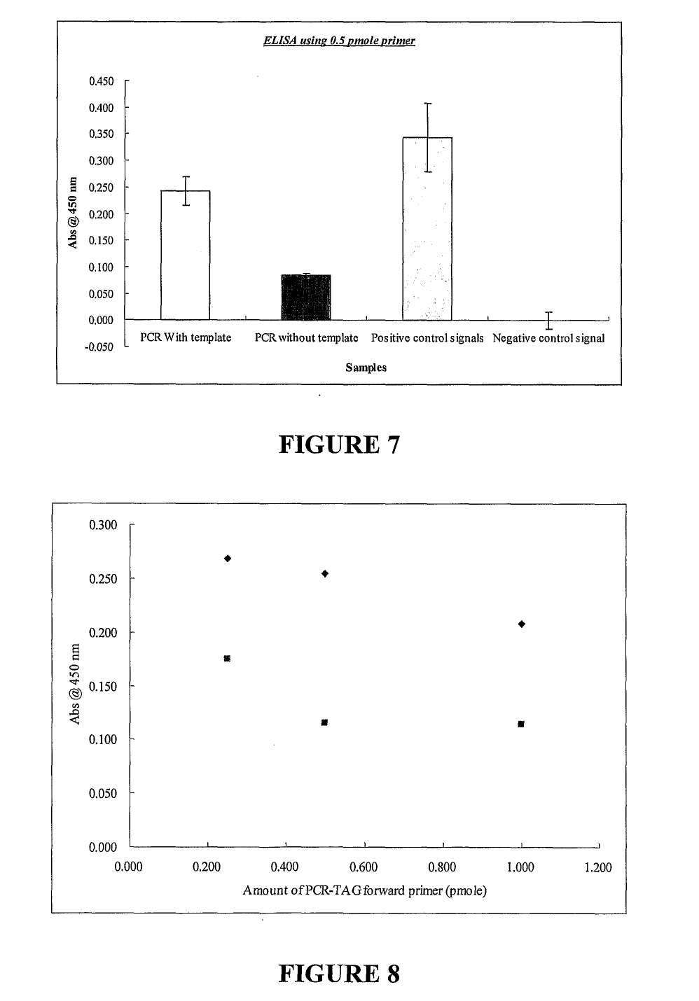

The results of this analysis, shown in FIG. 7, indicate that the assay can sensitively detect H7N7 influenza A cDNA with good signal to noise over background using end an point detection step.

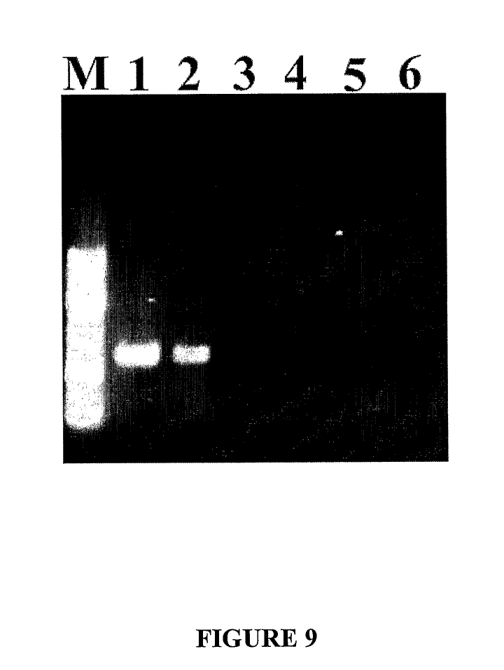

FIG. 8 shows the results of a titration experiment to determine the optimal amount of PCR-TAG primer in the assay described above. These results indicate that the optimal amount of PCR-TAG primer is approximately 0.5 pmole in 50 μL. The PCR products amplified using this method had the expected size, as determined by agarose gel electrophoresis and ethidium bromide staining (see FIG. 9).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A method for analysing a target nucleic acid sequence in a test sample, the method comprising:
combining in a reaction vessel:
(1) a capture oligonucleotide (e.g., immobilized or free in solution) that does not hybridize to the target nucleic acid sequence;
(2) a signaling oligonucleotide that provides a detectable signal and that hybridizes to the capture oligonucleotide;
(3) at least one chimeric oligonucleotide that comprises:
(a) a first targeting sequence that hybridizes to a subsequence of the target nucleic acid sequence; and
(b) a capturable sequence that hybridizes to a sequence selected from:
(i) the capture oligonucleotide; or
(ii) the signaling oligonucleotide
(4) at least one cooperating oligonucleotide that comprises a second targeting sequence that hybridizes to a sequence selected from:
(a) a different subsequence of the target nucleic acid sequence than the subsequence to which the first targeting sequence hybridizes; or
(b) a subsequence of a complementary strand of the target nucleic acid sequence, or
(c) at least one chimeric oligonucleotide; and
(5) a test sample comprising nucleic acid;
subjecting the contents of the reaction vessel to a nucleic acid processing reaction to form a reaction product if the target nucleic acid sequence is present in the test sample, wherein the reaction product thus formed comprises a first strand comprising at least one chimeric oligonucleotide as well as a second strand comprising at least one cooperating oligonucleotide, or extension product thereof, that blocks the hybridization of the capturable sequence of the chimeric oligonucleotide to the capture oligonucleotide, thereby allowing the signaling oligonucleotide to hybridize to the capture oligonucleotide; and
detecting the detectable signal from the signaling oligonucleotide which indicates the presence or amount of the target nucleic acid sequence in the test sample.

2. A method according to claim 1, wherein the nucleic acid processing reaction is a polymerization-dependent nucleic acid processing reaction.

3. A method according to claim 1, which comprises:
a) hybridizing the targeting sequence of the chimeric oligonucleotide to the target nucleic acid sequence to form a first hybrid wherein the target nucleic acid sequence extends in a 3′ to 5′ direction beyond the 3′ terminal nucleotide of the chimeric oligonucleotide to define a non-hybrid portion of the target nucleic acid sequence;
b) extending the chimeric oligonucleotide of the first hybrid in the presence of a polymerization agent and nucleotide precursors using the non-hybrid portion of target nucleic acid sequence as a template, to form a first duplex comprising a first extension product and the target nucleic acid sequence;
c) denaturing the first duplex to free the target nucleic acid sequence from the first extension product;
d) hybridizing the cooperating oligonucleotide with the first extension product to form a second hybrid wherein the first extension product extends in a 3′ to 5′ direction beyond the 3′ terminal nucleotide of the cooperating oligonucleotide to define a non-hybrid portion of the first extension product; and
e) extending the cooperating oligonucleotide of the second hybrid in the presence of a polymerization agent and nucleotide precursors using the first extension product as a template, to form a reaction product comprising the first extension product and a second extension product that is complementary to the first extension product.

4. A method according to claim 3, wherein steps a) to e) are repeated one or more times.

5. A method according to claim 3, wherein the polymerization agent is a primer dependent DNA polymerase.

6. A method according to claim 3, wherein the polymerization agent in step b) is a primer dependent reverse transcriptase.

7. A method according to claim 1, wherein the polymerization-dependent nucleic acid processing reaction comprises:
i) hybridizing a first targeting sequence of a circularizable first cooperating oligonucleotide to a first subsequence of the target nucleic acid sequence to form a first hybrid, wherein the 3′ nucleotide of the first cooperating oligonucleotide is complementary to the 5′ nucleotide of the first subsequence;
ii) hybridizing a second targeting sequence of the first cooperating oligonucleotide to a second subsequence of the target nucleic acid sequence, wherein the second subsequence is located adjacent to the first subsequence, to form a second hybrid,
iii) ligating the first and second targeting sequences of the first cooperating oligonucleotide in the presence of the 5′ nucleotide of the first subsequence and a ligation agent to form a first duplex comprising the first and second subsequences of the target nucleic acid sequence and a ligation product that comprises the first and second targeting sequences of the first cooperating oligonucleotide in a circularized form,
(iv) denaturing the first duplex to free the ligation product from the target nucleic acid,
(v) hybridizing a chimeric oligonucleotide with the ligation product to form a third hybrid;
(vi) extending the chimeric oligonucleotide of the third hybrid in the presence of a polymerization agent and nucleotide precursors using the ligation product as a template, to form a first extension product,
(vii) hybridizing a second cooperating oligonucleotide to the first extension product to form a fourth hybrid, and
(viii) extending the second cooperating oligonucleotide of the fourth hybrid in the presence of a polymerization agent and nucleotide precursors using the first extension product as a template, to form a reaction product comprising the first extension product and a second extension product that is complementary to the first extension product.

8. A method according to claim 1, wherein the nucleic acid processing reaction is a ligase-dependent nucleic acid processing reaction:

9. A method according to claim 3, which comprises:
1. hybridizing a first targeting sequence of a chimeric oligonucleotide to a first subsequence of the target nucleic acid sequence to form a first hybrid, wherein the 3′ nucleotide of the chimeric oligonucleotide is complementary to the 5′ nucleotide of the first subsequence;
2. hybridizing the second targeting sequence of a first cooperating oligonucleotide to a second subsequence of the target nucleic acid sequence, wherein the second subsequence is located adjacent to the first subsequence, to form a second hybrid;
3. ligating the chimeric oligonucleotide with the first cooperating oligonucleotide in the presence of the 5′ nucleotide of the first subsequence and a ligation agent to form a first duplex comprising the first and second subsequences of the target nucleic acid sequence and a ligation product that comprises both the chimeric oligonucleotide and the first cooperating oligonucleotide;
4. denaturing the first duplex to free the ligation product from the target nucleic acid; and
5. hybridizing a second cooperating oligonucleotide to the chimeric oligonucleotide and first cooperating sequence portions of the ligation product to form a reaction product.

10. A method according to claim 3, which comprises:
a. hybridizing a first targeting sequence of a first chimeric oligonucleotide to a first subsequence of the target nucleic acid sequence to form a first hybrid, wherein the 3′ nucleotide of the first chimeric oligonucleotide is complementary to the 5′ nucleotide of the first subsequence and the 5′ nucleotide of the capturable sequence is non-ligatable;
b. hybridizing a second targeting sequence of a second chimeric oligonucleotide to a second subsequence of the target nucleic acid sequence, which second subsequence is adjacent to the first subsequence, to form a second hybrid; wherein the 5′ nucleotide of the capturable sequence is non-ligatable;
c. ligating the first chimeric oligonucleotide with the second chimeric oligonucleotide in the presence of the 5′ nucleotide of the first subsequence and a ligation agent to form a first duplex comprising the first and second subsequences of the target nucleic acid sequence and a ligation product that comprises both the first chimeric oligonucleotide and the second chimeric oligonucleotide;
d. denaturing the first duplex to free the ligation product from the target nucleic acid; and
e. hybridizing a cooperating oligonucleotide to the first and second chimeric oligonucleotides of the ligation product to form a reaction product.

11. A method according to claim 3, which comprises:
i. hybridizing a first targeting sequence of a first chimeric oligonucleotide to a first subsequence of the target nucleic acid sequence to form a first hybrid, wherein the first chimeric oligonucleotide comprises a capturable sequence that is capable of hybridizing to the capture oligonucleotide and the 3′ nucleotide of the first chimeric oligonucleotide is complementary to the 5′ nucleotide of the first subsequence;
ii. hybridizing a second targeting sequence of a second chimeric oligonucleotide to a second subsequence of the target nucleic acid sequence, which second subsequence is adjacent to the first subsequence, to form a second hybrid; wherein the first chimeric oligonucleotide comprises a capturable sequence that is capable of hybridizing to the signaling oligonucleotide;
iii. ligating the first chimeric oligonucleotide with the second chimeric oligonucleotide in the presence of the 5′ nucleotide of the first subsequence and a ligation agent to form a first duplex comprising the first and second subsequences of the target nucleic acid sequence and a reaction product that comprises both the first chimeric oligonucleotide and the second chimeric oligonucleotide; and
iv. denaturing the first duplex to free the reaction product from the target nucleic acid.

12. A method according to any one of claims 9 to 11, wherein steps 1 to 5 or a to e or i to iv are repeated one or more times.

13. A kit comprising:
(1) a capture oligonucleotide (e.g., immobilized or free in solution) that does not hybridize to the target nucleic acid sequence;
(2) a signaling oligonucleotide that provides a detectable signal and that hybridizes to the capture oligonucleotide;
(3) at least one chimeric oligonucleotide that comprises:
(a) a first targeting sequence that hybridizes to a subsequence of the target nucleic acid sequence; and
(b) a capturable sequence that hybridizes to a sequence selected from:
(i) the capture oligonucleotide; or
(ii) the signaling oligonucleotide
(4) at least one cooperating oligonucleotide that comprises a second targeting sequence that hybridizes to a sequence selected from:
(a) a different subsequence of the target nucleic acid sequence than the subsequence to which the first targeting sequence hybridizes; or
(b) a subsequence of a complementary strand of the target nucleic acid sequence, or
(c) at least one chimeric oligonucleotide

14. A kit according to claim 13, further comprising (5) one or more polymerization and/or ligation agents.

15. A kit according to claim 13 or claim 14, wherein any one or more of components (1) to (5) are in lyophilized form.

16. A kit according to claim 13 or claim 14, wherein any two or more of components (1) to (5) are in the form of a mixture.

17. A kit according to claim 13 or claim 14, wherein any two or more of components (1) to (5) are in separate containers.

18. A kit according to claim 13, wherein the capture oligonucleotide is immobilized on a solid surface.

19. A kit according to claim 13, wherein the capture oligonucleotide is immobilized on the surface of a microparticle or bead.

20. A kit according to claim 13, wherein the capture oligonucleotide is immobilized on the surface of a nanowire.

21. A kit according to claim 13, wherein the capture oligonucleotide is immobilized on the surface of a reaction vessel.

22. A kit according to claim 13, wherein a plurality of capture oligonucleotides is immobilized in the form of a capture oligonucleotide array.

23. A kit according to claim 22, wherein the array is a solid phase array.

24. A kit according to claim 22, wherein the array is a liquid phase array.

25. A kit according to claim 13, wherein any one or more of components (1) to (4) are provided in a reaction vessel.

26. A kit according to claim 25, wherein the components that are present are preoptimized for conducting a method according to any one of claims 1 to 12.

27. A kit according to claim 26, wherein in order to conduct the method, the end user adds to the reaction vessel at least one of a nucleic acid sample, a nucleic acid processing enzyme, a chimeric oligonucleotide and a cooperating oligonucleotide.
